(19) United States
(12) Patent Application Publication
(10) Pub. No.: US 2020/0237378 A1
(43) Pub. Date: Jul. 30, 2020

(54) SHAPE MEMORY POLYMER-BASED DEVICES AND METHODS OF USE IN TREATING INTRACORPOREAL DEFECTS

(71) Applicant: The Board of Regents of the University of Oklahoma, Norman, OK (US)

(72) Inventors: Yingtao Liu, Norman, OK (US); Chung-Hao Lee, Norman, OK (US); Bradley Bohnstedt, Oklahoma City, OK (US); Robert Kunkel, Cranberry Township, PA (US)

(21) Appl. No.: 16/751,964

(22) Filed: Jan. 24, 2020

Related U.S. Application Data
(60) Provisional application No. 62/796,876, filed on Jan. 25, 2019.

(51) Int. Cl. A61B 17/12; A61L 31/02; A61L 31/14

(52) U.S. Cl. CPC A61B 17/12195; A61B 17/12113; A61B 2017/00871; A61L 31/14; A61L 31/024

(57) ABSTRACT

A novel shape memory polymer (SMP)-based device for surgical treatment of an intracorporeal defect (e.g., a void or anomaly) such as an intracranial aneurysm or fistula. In at least one non-limiting embodiment, the SMP device is a 3D-printed SMP material sized to specifically fit and thus occlude an intracranial aneurysm (ICA). The SMP device may be delivered to the intracorporeal defect via a catheter having a heating mechanism wherein the SMP device is raised above its glass transition temperature as it is deployed, causing the SMP device to return to its permanent shape after it is deployed into the intracorporeal defect. SMP device delivery systems that include the SMP devices, as well as methods of making and using the devices and systems, are also disclosed.

### CROSS REFERENCE TO RELATED APPLICATIONS/INCORPORATION BY REFERENCE STATEMENT

This application claims benefit under 35 USC § 119(e) of provisional application U.S. Ser. No. 62/796,876, filed Jan. 25, 2019. The entire contents of the above-referenced application are hereby expressly incorporated herein by reference.

### BACKGROUND

Stroke is a time-sensitive, medical emergency, a leading cause of serious, long-term disability, and the fifth leading cause of death (6% of all deaths) in Oklahoma in 2012. In the past two decades, endovascular therapy with Guglielmi detachable coils (GDCs) has become a well-received, minimally invasive technique for treating intracranial aneurysms (ICAs), also known as cerebral aneurysms. GDC-based embolization therapy, which aims at excluding the aneurysmal sac and neck from the cerebral circulation by means of complete and lasting occlusion, has been considered as an alternative to traditional surgical clip ligation associated with higher procedural mortality. However, recent studies have shown that there are still emerging clinical challenges in endovascular coil embolization, primarily aneurysmal recanalization and incomplete occlusion. In addition, more challenging clinical situations include the management and treatment of wide-necked aneurysms (with an unfavorable sack-to-neck ratio≈1.0) and large aneurysms (diameter d>10 mm), due to their complex 3D geometry for achieving complete occlusion. Despite the tremendous evolution of embolic techniques for treating these problematic aneurysms, issues of their relatively low packing density (occupying only 26%-33% of the aneurysm's volume) and low complete occlusion rates (˜60%-70%) still remain elusive.

Shape memory polymer (SMP) has been successfully used for brain aneurysm treatment in animal studies. SMP-based medical devices have been developed for the purposes of clot removal, aneurysm occlusion, and vascular stenting. In particular, SMPs have been designed to achieve aneurysm occlusion using four different approaches with a thermal triggering mechanism for device deployment: (i) coating on platinum coils, (ii) SMP-based embolic coils, (iii) SMP foams coupled with metallic or polymeric stents, and (iv) SMP stents. However, no SMP device has been made available clinically that can completely solve the deficiencies of the GDC-based embolization therapy. The focus of the present disclosure is to address and resolve these clinical and technical challenges.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

Several non-limiting embodiments of the present disclosure are hereby illustrated in the appended drawings. It is to be noted, however, that the appended drawings only illustrate several embodiments and are therefore not intended to be considered limiting of the scope of the present disclosure.

FIG. 1 is a top-view schematic of an apparatus for use in the synthesis of a shape memory polymer.

FIG. 2A shows a shear storage modulus analysis of twelve (12) SMP monomer compositions as directly measured from DMA tests.

FIG. 2B shows the tan (δ) curves as derived from the DMA testing results of FIG. 2A for determining the T_{g} of each SMP monomer composition.

FIG. 3 shows the TGA results, demonstrating the decomposition of the SMP specimen with increasing temperature, for all twelve (12) SMP monomer compositions of FIG. 2A.

FIG. 4 shows the DSC results used for determinations of the T_{g} for each SMP monomer composition of FIG. 2A.

FIG. 5 shows the mean±SEM of the failure stress (squares) and failure strain (circles) for all twelve (12) SMP monomer compositions of FIG. 2A (n=2) under uniaxial tension testing (T_{g}+10° C.).

FIG. 6A shows the representative cyclic mechanical testing results (SMP3) when tested at 50% of the observed failure strain (T_{g}+10° C.) showing the relaxation trend in the peak stress with an increasing number of cycles.

FIG. 6B shows the increase in the cumulative stress reduction.

FIG. 6C shows the convergence of the elastic modulus with an increasing number of cycles.

FIG. 7 shows the mean±SEM of the recovery testing time for a representative SMP composition (SMP3, n=3) showing the consistent trend of reduced recovery time with an increased temperature.

FIG. 8 contains representative experimental photos of the recovery testing for three representative SMP compositions (SMP3, SMP7, and SMP11) at defined time increments (t=0 sec, t=2 sec, t=4 sec, and t=6 sec), demonstrating the observed trend of a decreasing recovery time with an increasing TEA content.

FIG. 9 shows (a) the shape recovery of an SMP beam when temperature is above T_{g}; (b) a DSC testing result showing the T_{g} of SMP is 36° C.; (c) a DMA testing result showing the T_{g} is 40° C.; and (d) a TGA testing result showing that the SMP starts to decompose at about 260° C.

FIG. 10 shows (a) a schematic of a fabrication mechanism of carbon nanotube-SMP (CNT-SMP) nanocomposites; (b) an SEM image of a pristine SMP foam; (c-e) SEM images of CNT-enhanced nanocomposites; and (f) the porous size distribution of an SMP foam.

FIG. 11 shows (a) the shape recovery of a pristine SMP foam on a hot plate of 60° C.; (b) the effects of ultrasonication time and CNT concentration on the electrical resistivity of nanocomposites; and (c) the surface temperature of CNT-SMP nanocomposites during the Joule-heating process via different electrical current magnitudes.

FIG. 12 shows the shape recovery of a compressed CNT-SMP nanocomposite during Joule-heating.

FIG. 13 shows (a) the brain image data of a subject with cerebral aneurysm highlighted on the sagittal plane (left) and on the coronal plane (right), and (b) the 3D geometry of the aneurysm environment in the region of interest with other arterial blood vessels shaded as reconstructed from the subject's image data.

FIG. 14 shows a characterization of mechanical properties of the synthesized SMPs with various molar ratios, such as the failure stresses and failure strains. T_{g} decreases from 86° C. with 0.0 TEA content to about 40° C. with 0.6 TEA content.

FIG. 15 shows (a) the cyclic tensile testing results with 30% tensile strain, which demonstrates strain recovery, initial hysteresis, and material recovery due to the re-arrangement of the underlying polymer chains, and (b) a comparison of the stress-strain behavior between test data (40% strain, 1^{st} cycle) and predictions by the Arruda-Boyce constitutive model (C₁=2.8 MPa, λ=3.5, and R²=0.97).

FIG. 16 shows (a) a photograph of a surgical aneurysm creation in a rabbit pilot study through nerve artery vein, and (b) an intravenous aortogram showing successful aneurysm creation, with the same technique applied to the aneurysm creation procedure.

FIG. 17 shows (a) a schematic of a simulated aneurysm model with idealized geometries of the thin-walled parent blood vessel and aneurysm for FE simulations, whereas real patient-specific arterial vessel environment is used from a subject's image data, and computational model results of (b) a structural domain, and (c) flow & heat transfer domains.

FIG. 18 shows a schematic of an in vitro flow loop system with a patient-specific phantom aneurysm environment integrated with particle image velocimetry (PIV) techniques for measuring flow pattern, which will provide direct validation data for FE hemodynamic predictions.

FIG. 19 shows (a) a schematic of an SMP-based device with a heating element and a housing component for experiments under simulated endovascular conditions, and (b) a schematic of the 3D printed aneurysm with a soft PDMS coating for protection.

FIG. 20 shows a schematic of an SMP-based device in the simulated artery (a) before releasing SMP wires to the simulated aneurysm, and (b) after the SMP wire is fully released in the aneurysm with temperature increased by the heated element above the glass transition temperature.

FIG. 21 shows an SMP specimen as it changes shape as heated to the glass transition temperature.

FIG. 22 shows the DMA temperature sweeps showing the Tg of the synthesized SMP.

FIG. 23 shows the DSC testing results of the SMP of FIG. 21.

FIG. 24 shows the TGA testing results of the SMP of FIG. 21.

FIG. 25 shows the SEM images of an SMP foam from two different layers.

FIG. 26 shows the shape recovery of an SMP foam in response to a direct thermal trigger.

FIG. 27 shows the shape recovery of the SMP foam of FIG. 26 in response to a Joule-heating trigger mechanism.

FIG. 28 shows one non-limiting embodiment in which an SMP foam is compressed into a wire or filament shape for delivery via a catheter.

### DETAILED DESCRIPTION

Before further describing various embodiments of the apparatus, compositions, and methods of the present disclosure in more detail by way of exemplary description, examples, and results, it is to be understood that the embodiments of the present disclosure are not limited in application to the details of apparatus, methods and compositions as set forth in the following description. The embodiments of the compositions and methods of the present disclosure are capable of being practiced or carried out in various ways not explicitly described herein. As such, the language used herein is intended to be given the broadest possible scope and meaning, and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the apparatus, compositions, and methods of the present disclosure have been described in terms of particular embodiments, it will be apparent to those of skill in the art that variations may be applied to the apparatus, compositions, and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

The present disclosure is directed to shape memory polymer (SMP)-based devices for surgical treatment of an intracorporeal defect (e.g., a void or anomaly) such as (but not limited to) an intracranial aneurysm or fistula in a subject. In at least one non-limiting embodiment, the SMP device is a 3D-printed SMP material manufactured as tailored to specifically fit and thus occlude an intracranial aneurysm (ICA). The SMP device may be delivered to the defect via a catheter equipped with a heating mechanism wherein the SMP device is raised above its glass transition temperature as it is deployed, causing the SMP device to return to its permanent shape after the device has been deployed.

As noted above, prior GDC-based embolization treatment results in low packing density of the aneurysm's volume and unsatisfactory complete occlusion rates. In addition, the prior technology is not capable of completely stopping blood circulation in aneurysms in the long run; the patient may still suffer from stroke attack due to aneurysm rupture after medical surgery. Current SMP-based medical devices are manufactured without considering each patient's unique aneurysm geometries and pathological conditions, and are most commonly produced using the polymer casting method and thus have not fully solved the problems characteristic of GDCs. In order to improve the brain occlusion performance and to best optimize the medical device for a patient's unique condition, patient-specific 3D-printed SMP devices, based on the patient's particular aneurysm geometry, have been developed herein to improve the packing density of the aneurysm's volume and increase complete occlusion rates. The SMP devices of the present disclosure can achieve better occlusion by means of fully occupying the space of the aneurysm or other biomedical defect.

In one non-limiting embodiment, the SMP-based medical device comprises: (i) a patient-specific 3D-printed SMP device to occlude an intracranial aneurysm, (ii) a thermal deployment mechanism (such as, but not limited to, an electrothermal deployment mechanism or a photothermal deployment mechanism) to release the SMP device into the aneurysm, and (iii) a catheter for delivery of the SMP device into the patient's arterial system. The thermal deployment mechanism may include conductive wires (e.g., constructed of carbon fibers or other suitable heat conductive material) for generating heat to increase the temperature of the SMP device before releasing it into the patient's aneurysm. Once the temperature of the SMP device reaches the glass transition temperature, the material will start recovering its shape to the original design geometry. In at least one non-limiting embodiment, the SMP material has been formulated to have a glass transition temperature that is slightly above the normal human body temperature for the release of the SMP device in the brain tissue environment. The SMP device may be manufactured using additive manufacturing technology (e.g., 3D printing) and the patient-specific aneurysm geometries obtained from the patient's medical images, such as (but not limited to) a computed tomography (CT) or magnetic resonance imaging (MRI) scan. The SMP-based technology of the present disclosure is not limited to ICA or endovascular embolization, and, in fact, the desired mechanical performance and shape changing feature can be judiciously achieved to treat suitable intracranial defects or other biomedical applications, such as (but not limited to) Kyphoplasty surgery in spinal compressive fractures. In other non-limiting embodiments, the SMP-based technology of the present disclosure can be used for hemorrhage control, for example (but not by way of limitation) in battlefield situations, for wound dressing and healing, and as a foam scaffold for tissue repair and tissue engineering.

In at least certain non-limiting embodiments, the SMP device can be made using syringe extrusion-based 3D-printing (a.k.a., direct ink writing). In this method, the SMP pre-polymer is pre-cured and extruded from a syringe. In certain non-limiting embodiments, a photo-induced implantation process is used, wherein an infrared (IR)-based laser is used to generate heat to activate shape recovery of the SMP device locally during implantation. The IR light is absorbed by the SMP device so that the temperature of the SMP device will increase above its glass transition temperature. In at least certain non-limiting embodiments, the permanent shape of the SMP can be optimized by using combined computer simulations and additive manufacturing. For example, patient-specific CT images are used to reconstruct the 3D geometries of the defect (e.g., aneurysm). Through image-based computational modeling, the shape that can best fit into the defect will be calculated so that the final SMP shape will be the best fit for the treated defect. Once the desired permanent shape is determined, the SMP will be 3D printed so that the customized SMP-based device will be fabricated, packaged, and used for surgical operation.

In at least one non-limiting embodiment, the SMP device is made of a porous sponge (i.e., an open-cell material comprising pores) instead of a solid wire.

For example, the SMP (sponge) device can have a porosity in a range of from about 75% to about 85%, and, therefore, the volume can be compressed by about 80% to about 90% without fracturing the SMP structure. Compression may be done, for example, at a temperature about 10° C. above its glass transition temperature to ensure that no structural damage is induced in the SMP. When the compressed SMP sponge (now in its temporary shape) is cooled back to room temperature, the compressed (temporary) shape will be maintained. The compressed SMP sponge can be in a wire shape, which can then be inserted via a catheter for delivery. Once the catheter reaches the aneurysm during surgery operation, the compressed SMP is released out of the catheter, placed within the aneurysm, and heated, and then the SMP will autonomously recover its shape to fill the aneurysm's volume.

In certain non-limiting embodiments, the porous SMP device has an open-cell microstructure (e.g., having an average porous size in the non-limiting range of from about 50 μm to about 300 μm), so that blood can still flood into and saturate the sponge. Chemicals can be coated on the outersurface of the device and on the internal walls of pores in the device, and blood can be cured and caused to clot, so that the aneurysm is fully filled by bio-safe solid materials. In non-limiting examples, the inner walls of an open-cell porous SMP device can be coated with one or more chemicals that can cause blood coagulation, including hemostatic agents such as (but not limited to) chitosan, chitin, zeolite, and kaolinite; the fibrin precursor combination of fibrinogen, thrombin, factor XIII, and calcium; and the active ingredients in coagulants such as (but not limited to) CELOX™ (Medtrade Products Ltd, Crewe, UK), QUICKCLOT® (Z-Medica, LLC, Wallingford, Conn.), HEMCON® (Tricol Biomedical, Inc., Portland, Oreg.), FastAct, BLEEDARREST® (Hemostasis, LLC, St. Paul, Minn.), QUICK RELIEF® (Biolife LLC, Sarasota, Fla.), AND TRAUMADEX® (Medafor, Inc., Minneapolis, Minn.) hemostatic products. The rapid blood coagulation can cause the solidification of voids in the porous SMP device, resulting in stiffened SMP foam and stopped blood flow into the aneurysms. The external surface of the porous SMP device can be coated with an anticoagulant, so that blood will not adhere thereto minimizing stiffening of the surface of the SMP device that could cut the wall of the aneurysm thereby causing rupture and internal blooding.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error for the composition, the method used to administer the composition, or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, percentage, temporal duration, and the like, is meant to encompass, for example, variations of ±20%, or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, or at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Further, all references to one or more embodiments or examples are to be construed as non-limiting to the claims.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

By “biologically active” is meant the ability of an active agent to modify the physiological system of an organism without reference to how the active agent has its physiological effects.

As used herein, “pure,” “substantially pure,” or “isolated” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer an organism to which the compositions of the present disclosure are applied and used, such as (but not limited to) a vertebrate or more particularly to a warm-blooded animal, such as (but not limited to) a mammal or bird. Non-limiting examples of animals within the scope and meaning of this term include dogs, cats, rats, mice, guinea pigs, chinchillas, rabbits, horses, goats, cattle, sheep, llamas, zoo animals, Old and New World monkeys, non-human primates, and humans.

“Treatment” refers to therapeutic treatments, such as (but not limited to) for treating an intracorporeal (biomedical) defect. The term “treating” refers to administering the treatment (e.g., the SMP device) to a patient for such therapeutic purposes, and may result in an amelioration of the condition or disease.

The term “intracorporeal defect” as used herein will be understood to include (but not be limited to) intracranial defects and anomalies. Non-limiting examples of an intracranial defect include a void and an aneurysm. Non-limiting examples of an anomaly include a fistula.

The term “effective amount” refers to an amount of an active agent which is sufficient to exhibit a detectable biochemical and/or therapeutic effect, for example without excessive adverse side effects (such as (but not limited to) toxicity, irritation, and allergic response) commensurate with a reasonable benefit/risk ratio when used in the manner of the present disclosure. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by a person of ordinary skill in the art using routine experimentation based on the information provided herein.

The term “ameliorate” means a detectable or measurable improvement in a subject's condition or a symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as (but not limited to) stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as (but not limited to) a partial decrease, reduction, inhibition, suppression, limit, control, or prevention in the occurrence, frequency, severity, progression, or duration, or inhibition or reversal of the condition (e.g., stabilizing), over a short or long duration of time (e.g., seconds, minutes, hours).

Certain non-limiting embodiments of the present disclosure are directed to a shape memory polymer (SMP) device that comprises an SMP material having a permanent shape, a temporary shape, and a glass transition temperature. When in the permanent shape, the SMP material has a specific three-dimensional (3D) geometry unique to a specific intracorporeal defect in a subject, such that the permanent shape of the SMP material will substantially conform to and fill a space in the specific intracorporeal defect in the subject when the SMP material is deployed into the specific intracorporeal defect at a temperature above the glass transition temperature of the SMP material.

In a particular (but non-limiting) embodiment, the intracorporeal defect is an aneurysm, such as (but not limited to) an intracranial aneurysm (ICA).

In a particular (but non-limiting) embodiment, the glass transition temperature is in a range from about 36° C. to about 46° C., such as (but not limited to) a range from about 37° C. to about 43° C.

In a particular (but non-limiting) embodiment, the 3D geometry of the permanent shape of the SMP material is obtained from computed tomography (CT) imaging of the specific intracorporeal defect in the subject.

In a particular (but non-limiting) embodiment, the SMP material is a 3D-printed SMP object.

In a particular (but non-limiting) embodiment, the SMP material is an open-cell material comprising pores, and the pores are coated with a blood coagulant.

In a particular (but non-limiting) embodiment, the SMP material has an external surface, and wherein at least a portion of the external surface is coated with a blood anticoagulant.

In a particular (but non-limiting) embodiment, the SMP material comprises Hexamethylene diisocyanate (HDI), N,N,N0,N0-tetrakis (hydroxypropyl) ethylenediamine (HPED), and Triethanolamine (TEA).

In a particular (but non-limiting) embodiment, the SMP material comprises a radio-opaque additive.

In a particular (but non-limiting) embodiment, the SMP material is a carbon nanotube (CNT)-SMP material.

Certain non-limiting embodiments of the present disclosure are directed to a method of treating an intracorporeal defect in a subject. The method comprises inserting any of the SMP devices disclosed or otherwise contemplated herein into the intracorporeal defect of the subject.

In a particular (but non-limiting) embodiment, the intracorporeal defect is an aneurysm, such as (but not limited to) an intracranial aneurysm (ICA).

Certain non-limiting embodiments of the present disclosure are directed to an SMP device delivery system that comprises any of the SMP devices disclosed or otherwise contemplated herein, wherein the SMP device is in its temporary shape. The SMP device delivery system also comprises a heating mechanism for raising the temperature of the SMP device to a temperature above the glass transition temperature of the SMP material before the SMP device is deployed into the specific intracorporeal defect of the subject.

In a particular (but non-limiting) embodiment, the heating mechanism is electrotherm al.

In a particular (but non-limiting) embodiment, the heating mechanism is photothermal.

In a particular (but non-limiting) embodiment, the heating mechanism is heat resistive.

In a particular (but non-limiting) embodiment, the SMP device delivery system further comprises a catheter for delivery of the SMP device into the specific intracorporeal defect in the subject. In certain non-limiting alternatives of this embodiment, the heating mechanism comprises a portion of a terminal end of the catheter.

Certain non-limiting embodiments of the present disclosure are directed to a method of delivering an SMP device into an intracorporeal defect in a subject. The method comprises inserting any of the SMP delivery systems disclosed or otherwise contemplated herein into the subject to deliver the SMP device into the intracorporeal defect.

In a particular (but non-limiting) embodiment, the intracorporeal defect is an aneurysm, such as (but not limited to) an intracranial aneurysm (ICA).

### EXAMPLES

Certain embodiments of the present disclosure will now be discussed in terms of several specific, non-limiting, examples. The examples described below will serve to illustrate the general practice of the present disclosure, it being understood that the particulars shown are merely exemplary for purposes of illustrative discussion of particular (but non-limiting) embodiments of the present disclosure only and are not intended to be limiting of the claims of the present disclosure. In particular, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example I

This work was focused on characterization of an aliphatic urethane-based SMP device. Twelve compositions of the SMP were synthesized, and their thermomechanical properties together with the shape recovery behavior were comprehensively investigated. Results showed that the SMPs experienced a significant decrease in the storage and loss moduli when heated above their glass transition temperature (32.3-83.2° C.), and that all SMPs were thermally stable up to 265° C. Moreover, the SMPs exhibited both composition-dependent stress relaxation and a decrease in the elastic modulus during cyclic loading/unloading. The shape recovery time was less than 11 seconds for all SMP compositions, which is sufficiently short for shape changing and recovery during embolization procedures. Several candidate compositions were identified which possess a glass transition temperature above normal human body temperature (37° C.) and below the threshold of causing tissue damage (45° C.). They also exhibit high material strength and low stress relaxation behavior, indicating their potential applicability to endovascular embolization of ICAs.

One form of SMP is an aliphatic polyurethane as synthesized using hexamethylene diisocyanate (HDI), N,N,N0,N0-tetrakis (hydroxypropyl) ethylenediamine (HPED), and Triethanolamine (TEA), and comprises two segments at the molecular level: (1) rigid and glassy segments which determine the permanent shape, and (2) amorphous segments which control the temporary shape. Currently, most biocompatible SMPs used for biomedical applications are thermally induced. When heated above the SMP's glass transition temperature (T_{g}), the amorphous segments of the SMP transition from a glassy state to a rubbery state, thereby allowing the polymer to be deformed under an external load. After cooling below T_{g} while under the external load, the temporarily compressed shape is obtained. When the temperature of the SMP increases above the T_{g}, the SMP then autonomously decompresses and returns to the original, programmed shape without external mechanical stimuli. Biomedical devices fabricated using SMPs can be introduced into a patient's body in a temporary, compressed shape and then be expanded on demand to their programmed shape. Since the shape recovery can be facilitated with a certain triggering mechanism, such as (but not limited to) increasing temperature, the release of the SMP device can be completed without additional complex surgical operations, but rather through a micro-catheter.

In this example, aliphatic urethane-based SMPs were synthesized and comprehensively characterized to investigate connections between the working temperature of the polymers and their mechanical behavior. Glass transition temperatures of each composition were identified using both the dynamic mechanical analysis (DMA) and the differential scanning calorimetry (DSC) tests. The thresholds for thermal degradation of each composition were determined using thermogravimetric analysis (TGA). Uniaxial cyclic and failure testing results were obtained and analyzed for differences in behavior among the different compositions.

Methods

Materials and SMP Synthesis

Hexamethylene diisocyanate (HDI, ≥99.0%), N,N,N0,N0-tetrakis (hydroxypropyl) ethylenediamine (HPED, ≥98.0%), and Triethanolamine (TEA, ≥99.0%) were purchased from Sigma-Aldrich and used as received for synthesizing the aliphatic urethane shape memory polymers. Twelve combinations of these three monomers were synthesized, with their respective SMP formulations given in Table 1.

TABLE 1
Percent Monomer Content, Monomer-Mixture Stirring Time, and
the Curing Heating Rate for Twelve SMP Monomer Compositions

| | | | | Stirring | Heating |
|---|---|---|---|---|---|
| | Monomer Content (%) | | | Time | Rate |
| ID No. | HDI | HPED | TEA | (seconds) | (° C./hour) |
| SMP1 | 53.5 | 46.5 | 0.0 | 150 | 30.0* |
| SMP2 | 53.9 | 44.5 | 1.6 | 170 | 29.6 |
| SMP3 | 54.3 | 42.5 | 3.2 | 200 | 29.2 |
| SMP4 | 55.1 | 38.4 | 6.5 | 225 | 26.4 |
| SMP5 | 56.0 | 34.1 | 9.9 | 240 | 25.2 |
| SMP6 | 56.9 | 29.7 | 13.4 | 255 | 23.6 |
| SMP7 | 57.8 | 25.1 | 17.1 | 270 | 21.1 |
| SMP8 | 58.8 | 20.4 | 20.8 | 285 | 18.5 |
| SMP9 | 59.7 | 15.6 | 24.7 | 310 | 15.9 |
| SMP10 | 60.7 | 10.6 | 28.7 | 330 | 12.5 |
| SMP11 | 61.8 | 5.4 | 32.8 | 350 | 9.6 |
| SMP12 | 62.3 | 2.7 | 35.0 | 445 | 8.5 |

*Suggested same heating rate for SMP curing in Wilson et al. (2007)

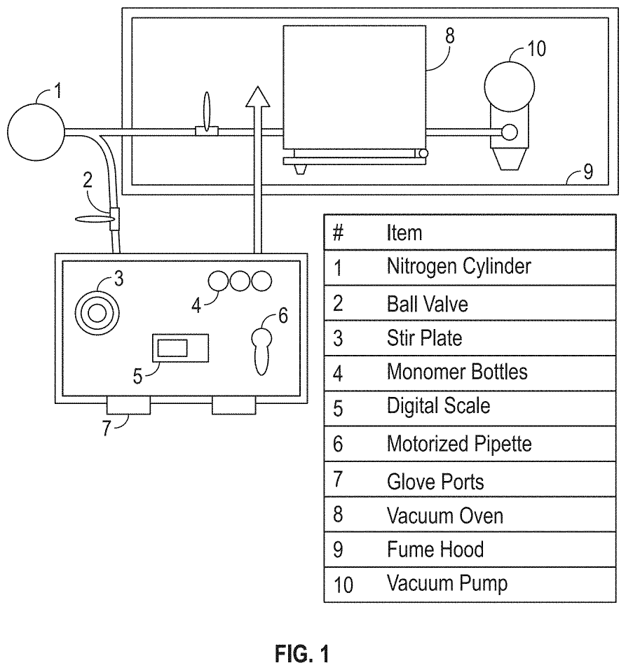

The molar ratios for each batch were sourced from Wilson et al., (Wilson, et al., “Shape memory polymers based on uniform aliphatic urethane networks.” J. Appl. Polym. Sci. (2007) 106:540-551) with modifications to the second and last compositions. All measurement and mixing procedures occurred within a nitrogen-filled glovebox to avoid moisture contamination of the monomers (FIG. 1). The glovebox received a steady flow of nitrogen through an inlet at the top of the rear panel and vented gas into a fume hood from an outlet at the bottom of the rear panel. This prevented air from entering the work space and removed any undesired moisture prior to the synthesis. Nitrogen flow could be redirected to the vacuum oven used later during synthesis via a set of ball valves.

In at least one non-limiting embodiment, the method comprises the steps of (i) measuring each of the monomers, (ii) mixing the monomers to form the polymer, (iii) disposing the mixture into the previously cast molds, and (iv) curing the mixture in the molds in a vacuum oven. Monomer weighing was performed using a Fisher brand motorized pipette filler (Thermo Fisher Scientific, Waltham, Mass.) and a digital scale (AWS-100, American Weigh Scales Inc., Cumming, Ga.). In brief, the HPED and TEA were measured in the same 100 mL glass beaker, while HDI was kept in a separate container until the stirring stage, where it was added to the mixture and stirred on a magnetic stirring plate. The mixture was stirred gently to avoid the introduction of gas bubbles into the liquid. Stirring continued until the mixture showed a sudden transition from translucent to uniformly clear. The time required to produce this transition increased as the ratio of HPED in the mixture decreased and the ratio of HDI increased (Table 1).

The procedures in Wilson et al., (op. cit.) suggested including an excess of 1-2% isocyanate (HDI). However, our early synthesis results were unsatisfactory, and the removal of this excess improved the success rate of our syntheses. A tendency of the mixtures to cure before degassing could take place, leaving large air bubbles in the resulting specimens, was also observed. Since the mixture of the monomers is an exothermic reaction, it was noticed that large batches of the mixture could generate adequate heat to act as a catalyst for the curing process. To avoid these scenarios, the size of each batch was limited to 16-18 g, and multiple small batches were mixed during a single synthesis procedure, rather than mixing the full volume all at once.

Once the mixture had sufficiently reacted, it was quickly removed from the glovebox, and the contents were poured into a set of silicone rubber molds—rectangular beams (45 mm×8 mm×1 mm) for glass transition-related characterizations and ASTM D638 Type V “dog bones” for uniaxial tensile mechanical testing (see below). Prior to the synthesis, two coats of mold release (Buehler 208186032) were applied to each specimen mold to minimize bubble generations due to any undesired interactions between the mixed monomers and the silicone rubber during curing. Then, specimen molds were placed in a vacuum oven (Being BOV-20), and five (5) vacuuming (−0.8 bar) and nitrogen purging steps were performed to create a nitrogen protected environment in the oven before degassing. A strong vacuum (−0.925 bar) was next induced using a vacuum pump for 10-12 minutes to remove gas bubbles trapped in the mixture (FIG. 1). For cases where multiple batches of mixture were used, each mold was filled half way with mixture, and an initial degassing step was performed while mixing the other batch. When the first degassing had finished, the rest of the space of the molds was filled, and then the above-mentioned vacuuming-purging and degassing steps were performed. An “overflow” section was included in the specimen molds to trap bubbles as introduced during the degassing procedure. The top few millimeters of each specimen could be polished off to leave a smooth finishing.

When curing the SMP specimens, the procedure in Wilson et al. (op. cit.) was followed with several modifications. The specimens were kept at room temperature for one hour, and then the temperature was increased at a steady rate to 130° C., where it was kept for another hour. The heating rate of temperature was proportional to the glass transition temperature (T_{g}) of the specimen being cured, to ensure that each SMP had an equal curing time before reaching its T_{g} (Table 1). During the curing process, a slow loss of vacuum potentially caused by the pressure increase associated with the heating was observed. To maintain a consistent vacuum, the oven was resealed in intervals of an increase of 7.5° C., by reestablishing the vacuum (−0.4 bar) and quickly purging the system with nitrogen. Upon completion of the curing step, the SMP specimens were carefully removed from the molds and stored in a vacuum desiccator (Bel-Art Lab) to ensure no moisture contamination occurred before subsequent thermo-mechanical characterization experiments.

Characterization of the Synthesized SMPs

The mechanical properties of shape memory polymers vary according to temperature, especially regarding T_{g}. To characterize these temperature-dependent mechanical properties with various polymer compositions, a series of thermomechanical tests were conducted, including the dynamic mechanical analysis (DMA), thermogravimetric analysis (TGA), differential scanning calorimetry (DSC), and uniaxial tensile tests considering failure and cyclic loading conditions, to pinpoint the T_{g} of the SMP compositions and to better understand their thermally-dependent mechanical behaviors.

Dynamic Mechanical Analysis (DMA)

Dynamic mechanical analysis (TA Q800) was used to measure the mechanical properties of synthesized SMPs. The SMP beam specimens were heated under a nitrogen atmosphere from 20° C. to 120° C. at a heating rate of 5° C./min and in the tension mode with a cyclic frequency of 1 Hz. DMA studies revealed the significant mechanical and thermal properties of the samples, such as (but not limited to) storage modulus, loss modulus, and glass transition temperature.

Thermogravimetric Analysis (TGA) and Differential Scanning Calorimetry (DSC)

Thermal analysis data were measured by both thermogravimetry (TA Q50, TA Instruments, New Castle, Del.) and differential scanning calorimetry (TA Q20, TA Instruments). All measurements were performed under a nitrogen environment. In brief, the thermal degradation behavior of the samples was recorded with heating from room temperature to 600° C. at a rate of 10° C./min. An in-house MATLAB (MathWorks, Inc., Natick, Mass.) program was used to determine the onset temperature of thermal degradation, which was used as a reference for the ensuing DSC measurements. The program performed a linear regression on a section of each specimen's TGA curve below T_{g} and another linear regression of the region on the TGA curve between 90% and 85% mass. The intersection of these two lines was determined to be the threshold of thermal stability. DSC measurements were carried out by: (1) heating from 20° C. to 160° C. at a rate of 5° C./min, (2) cooling to 20° C. at 50° C./min, (3) maintaining for 3 min at 20° C., and then (4) repeating the above procedures. DSC studies revealed the significant thermal properties of the samples, such as (but not limited to) the glass transition temperature. All the DSC data presented in this study were from the second heating cycle.

Mechanical Testing of the SMPs

Before performing tensile and cyclic testing on the SMP “dog-bone” specimens, the overflow region was removed to produce a clean finish on both sides of the specimen and eliminate imperfections. The samples were polished using a custom designed and 3D-printed mount on a rotary polishing machine (LaboPol-5, Struers Inc., Cleveland, Ohio). Once polished, the width and thickness of the testing region were measured three times each and averaged. Tensile failure testing was conducted using a uniaxial tensile testing system (Instron 5969, Instron, Norwood, Mass.). Double-sided padded tape was applied to both sides of each gripping region before mounting to prevent slippage during testing.

Failure testing was conducted at 10° C. above the T_{g} of each specimen in a temperature regulated environment on the Instron device. The specimens were mounted in three steps. First, the base of the sample was clamped into the bottom set of grips and allowed to heat up to the temperature of the testing environment. Second, the top section was clamped into the top set of grips, and the distance between the two grips was measured with a digital caliper. After measuring the distance between the grips, the extension reading on the Instron was zeroed, and as the sample returned to testing temperature, the grip positions were adjusted to keep the measured load as close to zero as possible. Finally, both sets of grips were tightened to make up for the relaxation of the SMP past its T_{g}. Once the sample reached testing temperature and the measured load was returned to zero, the extension measured by the Instron testing machine was added to the previously measured length, and the extension was zeroed once again. This value was recorded as the initial length of the specimen. Upon starting the test, the specimens were subjected to a displacement of 2 mm/min until failure. Five failure tests were completed per specimen, and the best three were selected for characterization purposes based on relative consistency of the elastic modulus and failure stress values.

The procedures for cyclic testing closely resembled those for failure testing. Another set of “dog-bone” specimens were tested at 10° C. above T_{g}, and the same three step mounting procedures were exercised as previously mentioned. For the cyclic tests, each sample underwent three cycles of preconditioning at 25% of the failure strain as determined during failure testing. After the preconditioning step, the samples underwent ten loading and unloading cycles of the previously determined 50% failure strain. Both preconditioning and cycling steps were carried out at the same strain rate of 2 mm/min as the failure tests.

Quantification of Shape Recovery Capability

The shape recovery function of the synthesized SMPs was investigated by bending a straight beam sample at a 180° angle, then measuring the time required for full recovery at various temperatures. In brief, the initial bend was achieved using a 3D printed mold. The beam was heated above its glass transition temperature, and then placed into the mold, where the specimen could cool and maintain its shape at the desired angle. To measure the recovery time, a video camera was placed directly above a beaker of water on a hot plate. The bent sample was held with forceps on a ring stand and swiftly lowered into the heated water bath, where the SMP specimen was fully recovered. The video was analyzed frame by frame to determine the elapsed time between any two specific angles of 45, 90, 135, 165, and 180 degrees. This procedure was conducted using water baths at T_{g}, T_{g}+5° C., and T_{g}+10° C. for each sample. Three repeated recovery tests were conducted at each of the above temperature levels, resulting in a total of nine (9) recovery time measurements for each SMP composition.

Results

DMA Results

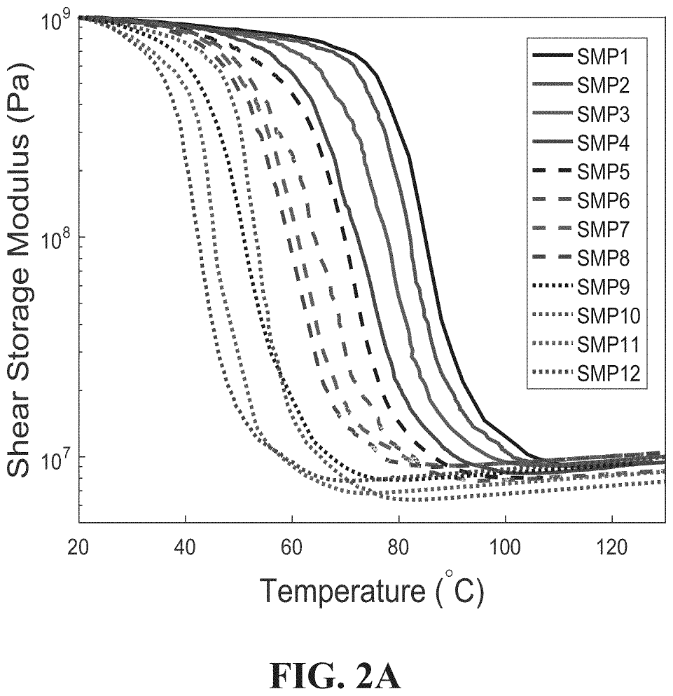

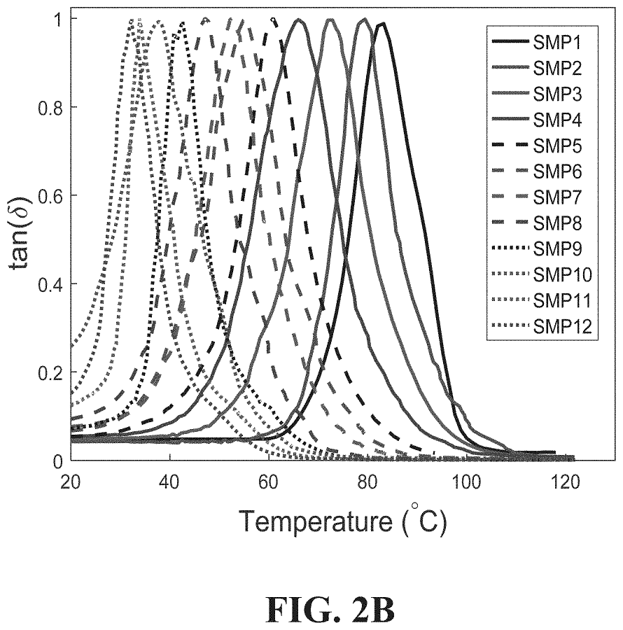

All SMP compositions showed a single steep transition in their shear storage modulus, each occurring at a different temperature threshold (FIG. 2A). A tan (δ) plot (FIG. 2B) was used to determine the glass transition temperature of each SMP composition. These values were taken at the peak of the tan (δ) plot and decreased monotonically from SMP1 to SMP12, ranging from 83.2° C. to 32.3° C. (Table 2). The storage moduli generally increased from SMP1 to SMP12; however, SMP10 exhibited exceptionally large values both above and below its glass transition temperature. Another factor which varied with the SMP composition was the change in the storage modulus from T_{g}−5° C. to T_{g}+15° C. With a few exceptions, the storage modulus of each specimen was reduced by a factor of 20-30 times its value at T_{g}−5° C. when raised to T_{g}+15° C. Shear modulus values at both temperatures tended to be larger for specimens nearer to SMP12, but there was not a consistent increase from one composition to another. A notable outlier is the shear modulus of SMP10 at T_{g}+15° C., which is exceptionally large compared to the other compositions.

The compositions between SMP9 and SMP11 possess transition temperatures between normal human body temperature (37° C.) and the threshold of tissue damage (45° C., >4-5 minutes), providing a desirable T_{g} range for allowing the polymers to remain functional within the body without causing any tissue damage due to the heating associated with shape change triggering.

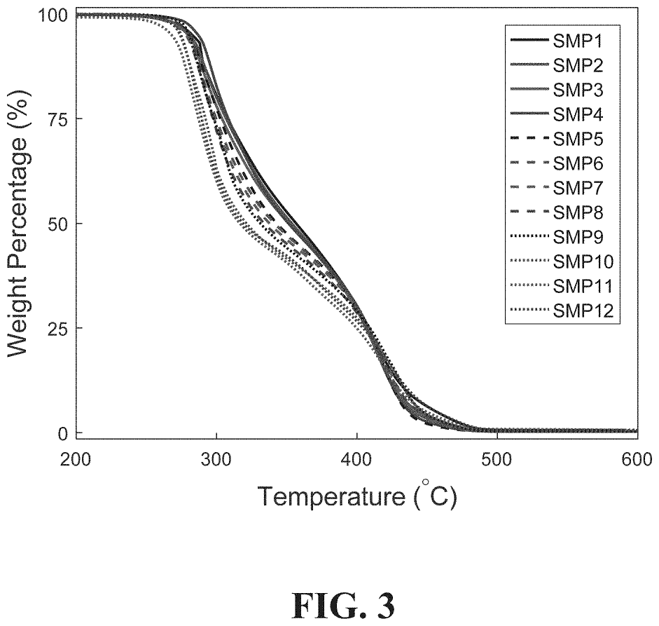

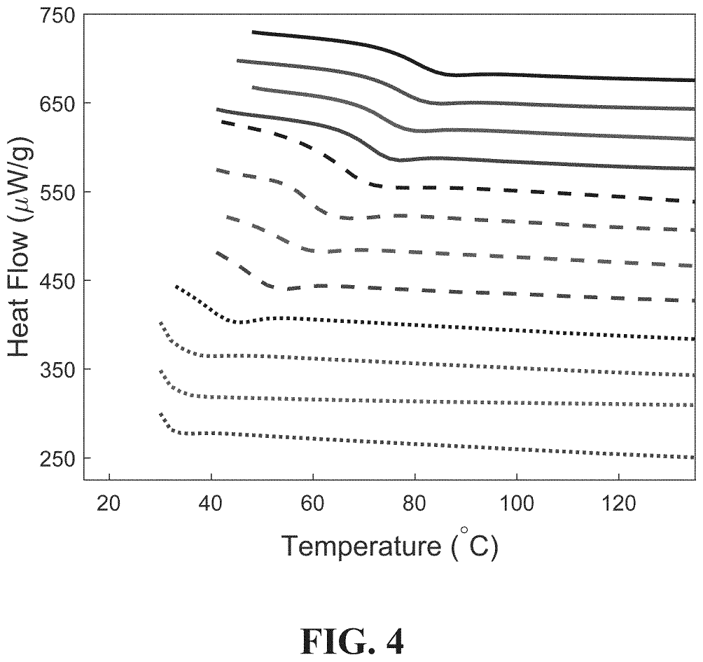

TABLE 2
Glass Transition Temperature (T_{g}) and Storage Modulus From the DMA Tests (FIGS.
2A-2B), T_{g} From the DSC Tests (FIG. 4), and the Temperature Levels Associated
With 90% and 50% Remaining Weights of the SMPs From the TGA Tests (FIG. 3)

| | | | | | TGA | | |
|---|---|---|---|---|---|---|---|
| | DMA | | | | Temperature | Temperature | Temperature |
| | | Storage | Storage | | (° C.) | (° C.) | (° C.) |
| | | modulus | modulus | | associated | associated | associated |
| | | at T_{g} − | at T_{g} + | DSC | with the onset | with 90% | with 50% |
| | T_{g} | 5° C. | 15° C. | T_{g} | of thermal | remaining | remaining |
| ID No. | (° C.) | (MPa) | (MPa) | (° C.) | degradation | weight | weight |
| SMP1 | 83.2 | 403.3 | 13.3 | 87 | 276.6 | 289.5 | 356.6 |
| SMP2 | 79.5 | 442.0 | 13.4 | 83 | 278.2 | 288.7 | 353.6 |
| SMP3 | 72.6 | 459.4 | 15.7 | 76 | 276.6 | 286.3 | 351.4 |
| SMP4 | 65.7 | 529.6 | 19.3 | 73 | 284.7 | 293.5 | 351.0 |
| SMP5 | 61.1 | 563.4 | 22.4 | 67 | 277.5 | 287.1 | 342.8 |
| SMP6 | 55.5 | 589.8 | 26.8 | 63 | 276.8 | 285.5 | 341.2 |
| SMP7 | 52.5 | 649.9 | 23.7 | 56 | 275.9 | 284.5 | 338.1 |
| SMP8 | 47.5 | 759.4 | 46.0 | 53 | 278.4 | 285.8 | 333.8 |
| SMP9 | 42.6 | 706.4 | 24.6 | 45 | 276.7 | 284.8 | 331.0 |
| SMP10 | 37.2 | 882.7 | 142.7 | 39 | 270.8 | 279.2 | 321.2 |
| SMP11 | 33.9 | 830.9 | 43.8 | 34 | 268.2 | 275.5 | 316.5 |
| SMP12 | 32.3 | 867.7 | 23.9 | 33 | 272.8 | 279.3 | 318.7 |

TGA Results

The TGA testing results (FIG. 3) show two major slopes occurring near 300° C. and 400° C., respectively. The distinction between these two slopes becomes more pronounced for SMP compositions closer to SMP12 that contain high percentages of the TEA. Values for the onset of thermal degradation were determined for each composition with values, showing no consistent trend, ranging from 268.2° C. to 284.7° C. (Table 2). The temperature at which each SMP composition degraded to 90% of its original weight was determined, and this value varied little between specimens, ranging from 275° C. to 293° C. Generally, this value increased from SMP1 to SMP12, but with an appreciable variation between individual compositions. The temperature required to degrade the SMPs to 50% weight varied more than the values for 90% degradation, ranging from 356.6° C. to 316.5° C.; however, these values showed a more uniform increase from SMP1 to SMP12.

DSC Results

The results of the DSC tests were used as a secondary means of determining the T_{g} of each SMP composition (FIG. 4 and Table 2). To extract these values, the local minimum of the resulting heat flow plots was used (FIG. 4), showing a monotonic decrease from SMP1 to SMP12. Such a monotonic decrease is also reflected in the T_{g} of the SMP compositions, ranging from 87° C. to 33° C. These T_{g} values from the DSC testing generally agree with the values determined using the tan (6) plot in the DMA tests (FIGS. 2A-2B). However, the T_{g} values determined using DSC analysis are consistently higher than those from DMA and tan (δ) analysis, but the difference is small enough to attribute to differences arising from the method of determination. A similar difference was observed in the analysis performed by Wilson et al. (op. cit.).

Uniaxial Tensile Testing Results

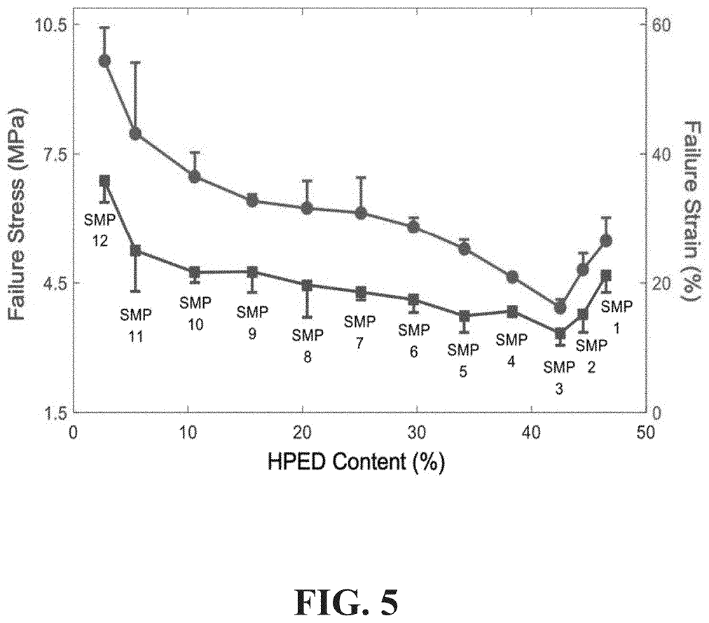

Under uniaxial tensile failure tests, the SMPs exhibited a sharp decrease in the failure stress and the failure strain from SMP1 to SMP3 and an increase in both failure stress and failure strain from SMP3 to SMP12 (FIG. 5 and Table 3). The trends in the data are nonlinear, with large increases near compositions SMP12 and SMP1 (FIG. 5). The maximum failure stress and strain occur at SMP12, with values of 6.88 MPa±0.29 MPa and 54.4%±2.97%, respectively. The minimum stress and strain occur at SMP3, with values of 3.34 MPa±0.16 MPa and 16.2%±0.72%, respectively. For most of the specimens, a decrease in both failure stress and strain was observed as the HPED content increased in the SMP composition.

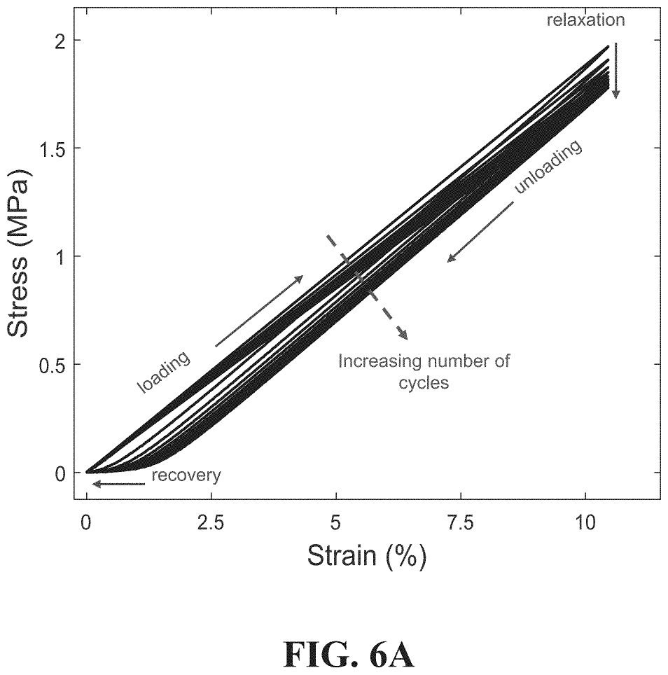

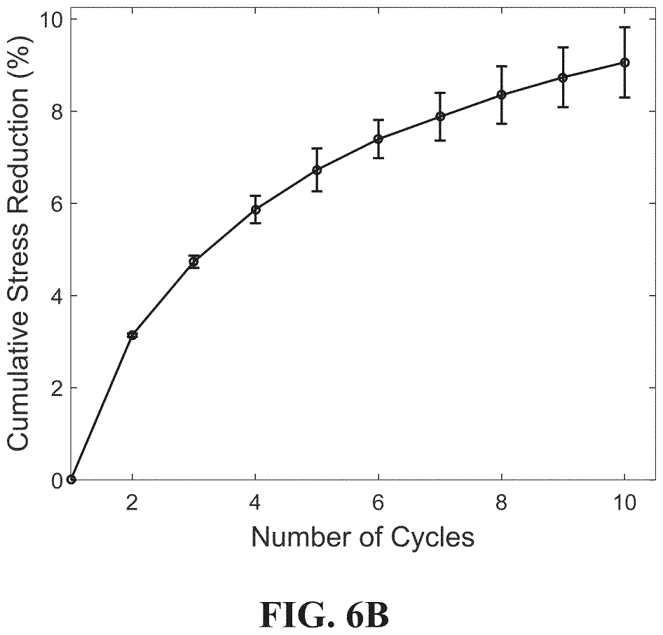

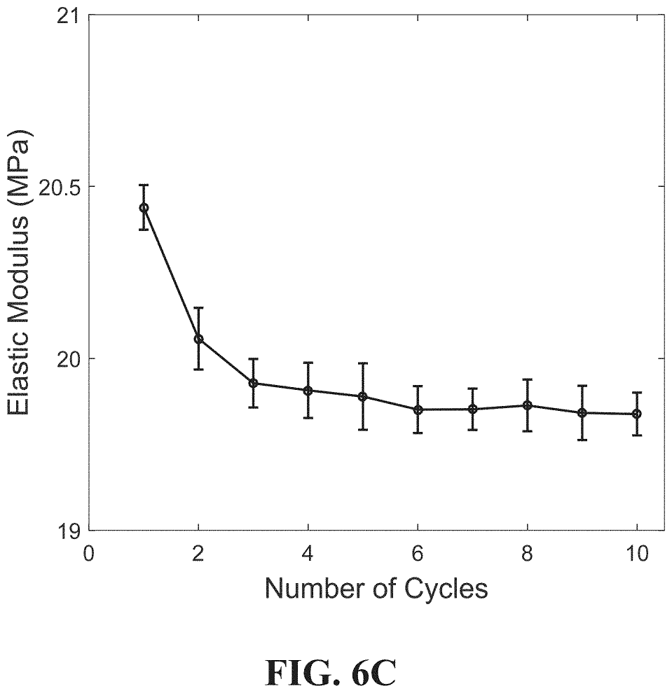

TABLE 3
Failure Stresses and Failure Strains From the Uniaxial Tensile Failure
Testing (FIG. 5) and the Stress Reductions and Calculated Elastic Modulus
From the Uniaxial Cyclic Tensile Testing (FIG. 6A-6C) for the Twelve
SMP Test Compositions. Tensile Tests Conducted at Tg + 10° C.

| | | | Uniaxial Cyclic Tensile Test | | |
|---|---|---|---|---|---|
| | Uniaxial Tensile | | Cumulative stress | | |
| | Failure Test | | reduction (%) | | Elastic |
| | Failure | Failure | 2^{nd} cycle | 10^{th} cycle | modulus |
| | stress | strain | w.r.t. | w.r.t. | at the 10^{th} |
| ID No. | (MPa) | (%) | 1^{st} cycle | 1^{st} cycle | cycle (MPa) |
| SMP1 | 4.68 ± 0.23 | 26.5 ± 2.11 | 7.66 ± 0.42 | 26.9 ± 3.93 | 22.58 ± 0.08 |
| SMP2 | 3.78 ± 0.21 | 22.1 ± 1.24 | 3.36 ± 0.98 | 7.88 ± 2.16 | 20.74 ± 0.52 |
| SMP3 | 3.34 ± 0.16 | 16.2 ± 0.72 | 1.83 ± 0.59 | 9.08 ± 4.58 | 18.97 ± 0.33 |
| SMP4 | 3.84 ± 0.07 | 20.9 ± 0.32 | 3.14 ± 0.03 | 9.06 ± 0.54 | 19.84 ± 0.04 |
| SMP5 | 3.74 ± 0.22 | 25.3 ± 0.76 | 3.30 ± 0.62 | 8.39 ± 1.66 | 18.80 ± 0.82 |
| SMP6 | 4.11 ± 0.17 | 28.7 ± 0.83 | 2.79 ± 0.03 | 6.64 ± 0.49 | 20.39 ± 1.48 |
| SMP7 | 4.29 ± 0.11 | 30.9 ± 3.14 | 2.28 ± 0.29 | 5.68 ± 0.37 | 18.50 ± 0.18 |
| SMP8 | 4.45 ± 0.43 | 31.6 ± 2.44 | 2.26 ± 0.25 | 6.70 ± 0.24 | 19.04 ± 0.77 |
| SMP9 | 4.76 ± 0.28 | 32.7 ± 0.58 | 0.41 ± 0.12 | 1.15 ± 0.04 | 18.34 ± 2.12 |
| SMP10 | 4.74 ± 0.14 | 36.5 ± 2.14 | 0.51 ± 0.09 | 7.42 ± 0.03 | 16.32 ± 0.52 |
| SMP11 | 5.25 ± 0.55 | 43.2 ± 6.29 | 0.80 ± 0.47 | 1.92 ± 1.36 | 15.26 ± 0.25 |
| SMP12 | 6.88 ± 0.29 | 54.4 ± 2.97 | 0.93 ± 0.06 | 3.34 ± 0.85 | 13.14 ± 0.31 |

As for the uniaxial tensile cyclic tests, the SMPs showed a noticeable relaxation behavior under cyclic tensile testing. This can be seen in the representative specimen (FIG. 6A). The relaxation behavior is different depending on the specimen composition, and it generally decreases from SMP1 to SMP12. Within individual specimens, the relaxation behavior followed a regular pattern (FIG. 6B), exhibiting large but decreasing relaxation during the first six cycles and then transitioning to uniform small relaxation during later cycles. The maximum reduction observed at the end of the tenth cycle was 26.9%±3.93% for SMP1, while the minimum was observed to be 1.15%±0.04% for SMP9. The elastic moduli of the SMPs were also affected by the cyclic loading, decreasing sharply during the first two cycles but remaining nearly constant after the fourth (FIG. 6C). The elastic modulus also varied with the SMP composition, with a gradual decrease from SMP1 to SMP12. SMP1 displayed the largest elastic modulus, with a value of 22.58 MPa±0.08 MPa, while SMP 12 displayed the smallest, with a value of 13.14 MPa±0.31 MPa (Table 3).

Shape Recovery Capability

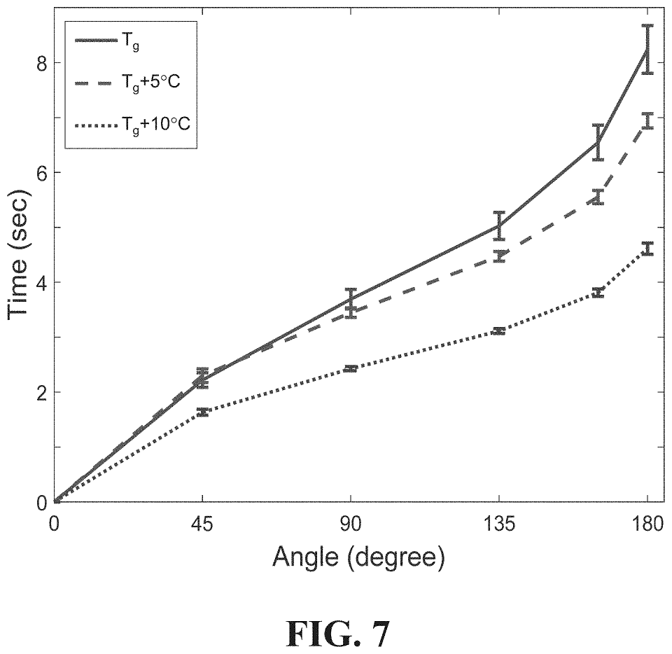

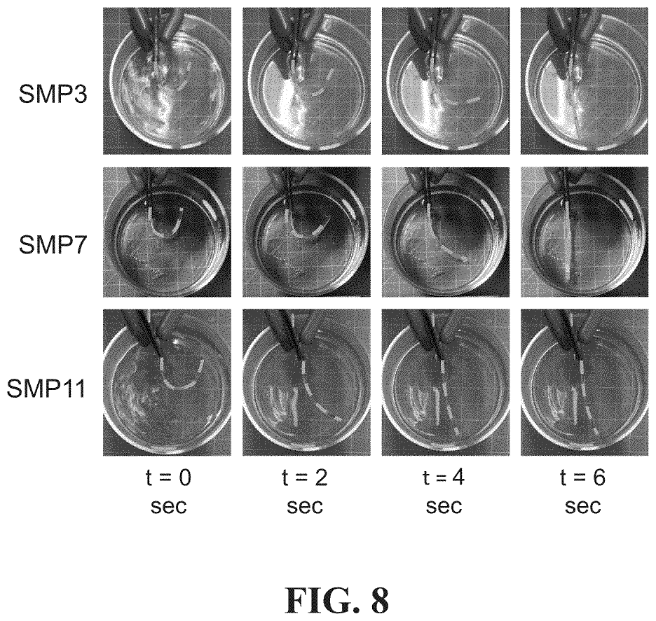

The SMPs showed a consistent temperature dependence in their shape recovery behavior, an example of which is shown in FIG. 7. Among individual specimens, the SMPs showed a slower recovery response between the initiation of the test and the first 45° of recovery, a fast, linear response between 45° and 135°, and a nonlinear deceleration as it approached a full 180° recovery. The results of the recovery tests indicated no significant trends in the recovery time with relation to the SMP composition. There was a tendency for specimens with a high TEA content (closer to SMP12) to recover faster than those with a high HPED content (closer to SMP1). However, several SMP compositions fell outside of this trend that it cannot be considered significant. FIG. 8 shows a direct comparison of the recovery test results at T_{g}+5° C. among three (3) selected SMP compositions (SMP3, SMP7, and SMP11).

Overall Findings and Relevance to Endovascular Embolization Treatment for ICAs

The thermomechanical characterization of the aliphatic urethane SMPs provided a closer look at the shift in material properties that occurs as each SMP reaches its T_{g}. The DMA results showed a single sharp transition in the shear storage modulus for all compositions (FIG. 2A). It was observed that this transition occurs at different temperature levels depending on the SMP composition, with higher glass transition temperatures corresponding to higher concentrations of HPED. In this Example, the glass transition temperature of the SMP specimen was determined from these transitions with SMP compositions tested, ranging from 83.2° C. to 32.3° C. In the context of implantable embolic devices, the SMP may possess a T_{g} above body temperature (i.e., above 37° C.) but below the threshold for tissue damage (about 45° C.). If the T_{g} is below body temperature, then the implant would constantly exist in a malleable state and not hold any one specific shape. However, at temperature levels greater than 45° C., bodily tissues can begin to take damage. This desired threshold falls within the observed T_{g} values, indicating that an aliphatic urethane SMP device can be synthesized by employing the presently disclosed techniques, which transitions at a temperature level suitable for applications in a body.

Moreover, uniaxial mechanical testing was conducted using the SMPs to determine their material strength and investigate how the strengths varied with composition. The failure test results demonstrated that higher values for both failure stress and strain occur in compositions with lower HPED contents, but the trend is nonlinear with significant variances at SMP3 (FIG. 5). Because of its irregular trends, this data will be difficult to use in a predictive manner, but it implies that there may be more complex changes associated with the SMP's composition than previously expected. With a wider range of compositions and larger sample sizes for each composition, future studies could identify trends which could allow fabrications of SMP-based biomedical devices with specific material strengths.

Cyclic tensile testing was performed to investigate changes in the behavior of the SMP under repeated loading. The two major properties that were investigated were the elastic modulus and the peak stress value at 50% failure strain (Table 3). GDC-based coils are designed to be left in the body for the remainder of a patient's lifetime, so it is important that the SMP materials used for this endovascular embolization application will not relax over time, resulting in the aneurysm recurrence. One behavior that the cyclic testing revealed was a noticeable reduction in the peak stress, with most of the reduction occurring during the first few cycles. This stress reduction reached a maximum value of 26.9%±3.93% in SMP1 with respect to the first cycle, and the next highest values fell near the range of 8%-9% for SMPs 2-5. The relaxation behavior, which is not a desirable quality in the context of a permanent embolization device, was more pronounced for SMPs containing more HPED contents. The compositions containing large quantities of TEA exhibited less relaxation, reaching values as low as 1.15%±0.04% for SMP9 and 1.92%±1.36% for SMP11. In addition, the elastic modulus also varied with cyclic loading, but only during the first few cycles of the test. The elastic modulus values decreased sharply during the first cycle, but quickly reached a constant value around the third or fourth cycle (FIG. 6A). Even though the changes in elastic modulus are small, it is desired to minimize any changes in material properties once the SMP is introduced into the body. In at least certain non-limiting embodiments, the embolization devices should undergo pre-cycling before implantation, minimizing the effects of initial relaxation when the device is administered.

Another important factor in designing an embolic device made from SMPs is the shape recovery behavior which occurs when the polymer transitions from a deformed state to its unstressed state. The recovery tests conducted in this Example focused on the time required for the SMP to recover its shape. For endovascular embolization of ICAs, a short recovery time of the SMP-based device enables the device to avoid the prolonged heating of body tissues during device deployment. The recovery behavior of the SMPs was shown herein to be temperature dependent, speeding up as temperatures increased past the T_{g}. At T_{g}+10° C., no composition took more than 10.3 seconds to fully recover from a 180° bend.

In at least certain non-limiting embodiments, the SMP devices of the present disclosure include radio-opaque additives to make them visible under x-ray-based fluoroscopy so that physicians can pinpoint their location and orientation during device deployment. Without such additives, urethane-based shape memory polymers are typically invisible to radiographic imaging techniques. Examples of such radio-opaque materials include, but are not limited to, tantalum and bismuth (III) oxychloride.

Thermal energy can be supplied to the SMP devices via diverse activation techniques, many of which are based on the indirect delivery of thermal energy to the material. These methods include, but are not limited to, Joule heating with the addition of conductive inclusions, optical heating achieved using wavelength specific dyes and a matching laser light source, and magnetic stimulation of nanoparticles. Another activation technique uses chemical interactions to lower the T_{g} of the SMP below ambient temperature, triggering the shape memory effect. This effect occurs slowly in polyurethane SMPs, and quickly in hydrogels, when the materials are exposed to water.

The results indicate that in SMP compositions closer to SMP12, the decreases in both maximum stress and elastic modulus with cyclic loading were not as prominent. Since any changes in material properties of the device after implantation tend to be detrimental, the SMP compositions near SMP12 are more desirable in the context of endovascular embolization treatment for ICAs.

### Example II

In this example, a highly porous carbon nanotube (CNT)-shape memory polymer (SMP) nanocomposite for the endovascular ICA treatment is presented. Pristine SMP foam is fabricated using a biologically safe and environmentally friendly sugar template method. A CNT-SMP nanocomposite foam is fabricated by infiltrating CNTs into pristine SMP foam in ethanol by ultrasonication. The porous nanocomposites are characterized to identify key parameters, such as (but not limited to) average pore size, density, porosity, and electrical resistivity. A resistive-heating mechanism can be used to trigger the shape recovery of the nanocomposites.

Materials

Chemicals were purchased from Sigma-Aldrich and used as received. Three monomers were used to synthesize the aliphatic urethane SMPs. The monomers used to synthesize SMP were: (i) Hexamethylene diisocyanate (HDI), (ii) N,N,N0,N0-tetrakis (hydroxypropyl) ethylenediamine (HPED), (iii) Triethanolamine (TEA). The molar composition ratio of the HDI, HPED, and TEA monomers was 1:0.05:0.6. Multi-walled carbon nanotubes (CNTs) (50-90 nm diameter, >95% carbon basis) improved the electrical conductivity of SMP nanocomposites. Ethanol was used as solvent to infiltrate CNTs into SMP foams.

Preparation of Solid SMP

To synthesize the solid SMPs, amounts of monomers of HPED, HDI, and TEA were measured and combined into a mixture which was mixed using a high-speed shear mixer for 3 minutes, and then cast into a “dog-bone” shape or rectangular molds for curing. The materials were degassed three times, and nitrogen was used to protect all the materials before and during curing using a vacuum oven. The temperature profile was first kept at room temperature for 60 minutes, followed by a ramp of 30° C. per hour up to 130° C., then followed by 1 hour at 130° C., and finally cooled back to room temperature naturally. The fully cured SMP samples were removed from the molds and saved for testing and characterization.

Preparation of SMP and Nanocomposite Foams

Porous and pristine SMP foams were synthesized using a sugar template assisted method. A sugar template was first manufactured by compressing a suitable amount of cane sugar (sucrose) into a silicon rubber mold. A slight amount of water was added to improve the formability of cane sugar. Once the sugar template was prepared, an appropriate amount of SMP monomers was measured and mixed by a high-speed shear mixer for 3 minutes, then the sugar template was merged into the mixed pre-polymer and kept in vacuum at −5° C. for 24 hours. The SMP monomers were fully cured following the heating procedure introduced above. The sugar template was dissolved in de-ionized water using a bath sonicator for 1 hour. The manufactured SMP foams were kept in a vacuum oven at 30° C. for 24 hours to fully eliminate all the humidity trapped in the SMP foams.

CNT-SMP nanocomposite foams were manufactured using the pristine SMP foam. An appropriate amount of CNTs was first dispersed in ethanol by mechanical shear mixing for 5 minutes and followed by bath sonication for 10 more minutes. Then a prepared SMP foam was submerged in the CNT-ethanol solution to infiltrate CNTs into the SMP foam using an ultrasonication bath. Finally, the open-cell CNT-SMP nanocomposite foams, with interconnected CNF network deposited on the porous wall, were dried and attained for testing. The detailed CNT concentration used during ultrasonication is shown in Table 4. CNT concentration in ethanol solution used in this Example was in the range of 0.001-0.006 g/ml. The sonication time was up to 50 minutes.

TABLE 4
CNT Concentration in Ethanol Solution
During Infiltration via Ultrasonication

| | CNT | Ethanol | CNT concentration |
|---|---|---|---|
| | (g) | (ml) | (g/ml) |
| 1 | 0.05 | 50 | 0.001 |
| 2 | 0.10 | 50 | 0.002 |
| 3 | 0.15 | 50 | 0.003 |
| 4 | 0.20 | 50 | 0.004 |
| 5 | 0.25 | 50 | 0.005 |
| 6 | 0.30 | 50 | 0.006 |

Experimental Characterization of Solid SMP

Dynamic mechanical analysis (DMA) was employed to determine the thermal transitions in the synthesized SMPs. Experiments were conducted using a TAQ800 dynamic mechanical analyzer. Rectangular beam samples were tested using tensile mode from 30° C. to 130° C. Differential scanning calorimetry (DSC) was employed to characterize the polymer's thermal properties, in particular to validate T_{g}. The comparison of T_{g} obtained from DMA and DSC can validate each other. Thermogravimetric analysis (TGA) tests were conducted to study the decomposition temperature and procedure of the synthesized SMP. The maximum allowable temperature of the SMP can be obtained from the TGA tests.

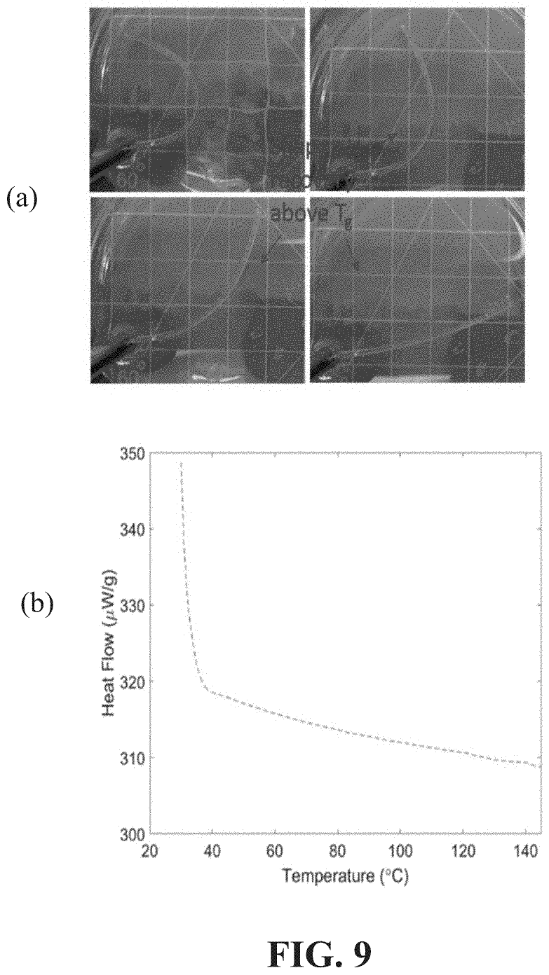

The characterization results of solid SMP are shown in FIG. 9, panels (a-d). The shape recovery capability of SMP is shown in FIG. 9, panel (a). When the beam sample is heated above the material's T_{g}, the beam sample autonomously rotated and recovered from the bend shape to the original straight beam shape. DSC and DMA tests showed that the T_{g} of the synthesized SMP is 36° C. and 40° C., respectively. TGA tests showed that the polymer begins decomposing at 260° C. Since this polymer will be utilized at or around body temperature, the polymer is safe during the shape recovery process.

Experimental Characterization of Synthesized SMP and CNT-SMP Nanocomposite Foam

Microstructure and morphology of pristine SMP and CNT-SMP nanocomposite foam were characterized by field-emission scanning electron microscope (SEM). The pore size and size distribution of the pristine SMP foam were determined using commercial software ImageJ (NIH, Bethesda, Md.) using five (5) SEM pictures.

The volume electrical resistivity of fabricated CNT-SMP nanocomposites was measured using the two-probe method. CNT-SMP nanocomposites were fabricated into cubic samples and clamped between two flat electrodes to measure the electrical resistance of the sample. The volume resistivity was calculated using Eqn. 1.

$$a ) ρ = R \frac{A}{l} \qquad ( Eqn . 1 )$$

where R is the electrical resistance, A is the cross-section area of the sample, and l is the height of the sample.

Results

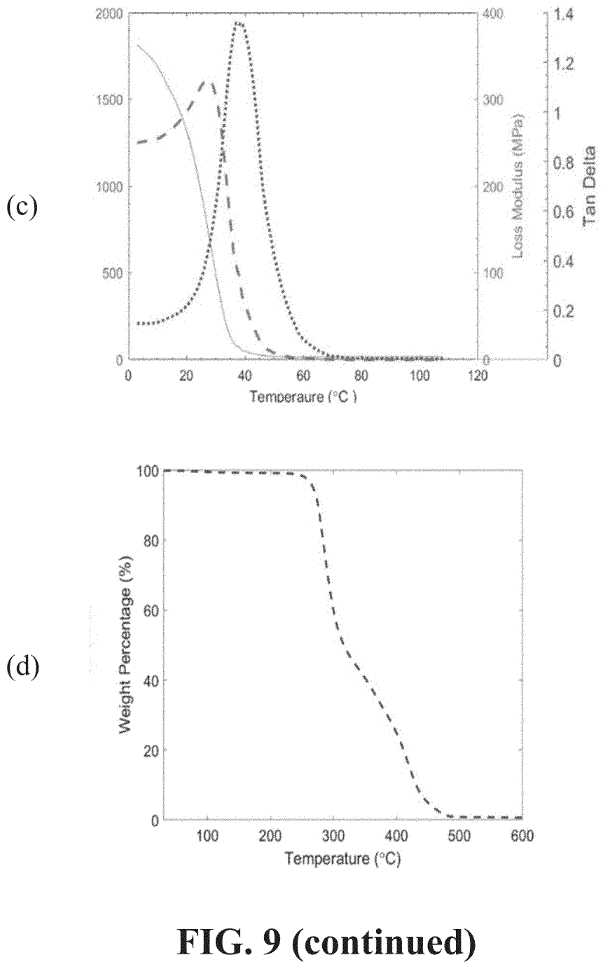

The mechanism of CNT infiltration is shown schematically in FIG. 10, panel (a). SMP pre-polymer was able to flow into the porous area in sugar templates since the curing time was significantly increased by keeping the mixed pre-polymer and sugar templates at low temperature. After 24 hours, the mixed pre-polymer was fully solidified, and sugar was able to be completely dissolved by de-ionized water, generating a highly porous microstructure of pristine SMP foam. CNTs were deposited on the porous walls when the pristine SMP foam and CNT nanoparticles were sonicated in an ethanol solution, forming a highly conductive network within SMP foam to enhance the electrical properties.

The porosity of manufactured pristine SMP foam was calculated using Eqn. 2, where P is the porosity, d_{f} is the density of SMP foam, and dₛ is the density of solid SMP. The weight and dimensions of five cubic samples were measured to calculate the density of solid SMP and SMP foam. The average density of solid SMP was 1.172 g/cm³, and the average density of pristine SMP foam was 0.168 g/cm³. Therefore, the porosity of the SMP foam reported in this Example was 85.7%.

$$P = 1 - \frac{{d}_{f}}{{d}_{s}} \qquad ( Eqn . 2 )$$

FIG. 10, panels (b-d) shows the SEM image of both pristine SMP foam and CNT-SMP nanocomposites. A highly porous, interconnected three-dimensional microstructure was obtained in the pristine SMP foam (FIG. 10, panel (b)). It was observed that all the sugar particles have been dissolved and removed from the SMP foam. No significant difference in the morphology and microstructure was observed from the six sides of SMP cubic samples. SEM images showed that CNTs were uniformly deposited within the SMP foam during sonication (FIG. 10, panels (c-e)). In particular, FIG. 10, panel (e) clearly demonstrated that CNTs were infiltrated during ultrasonication and deposited on the SMP wall to form an electrically conductive network, without forming significant CNT agglomerations. The conductive CNT network improved the electrical conductivity of the SMP foam, resulting in the resistive-heating (also known as Joule-heating) triggered shape recovery of CNT-SMP nanocomposites. In addition, ultrasonication did not deteriorate the SMP foam in the ethanol solution. The average pore size of pristine SMP foam was 253±140 μm (FIG. 10, panel (f)). It is noteworthy that the pore size and porosity of the SMP foam strongly depends on the structure of the sugar template. Therefore, both the pore size and porosity of the SMP foam can be adjusted by using different types and sizes of sugar particles for the fabrication of sugar templates.

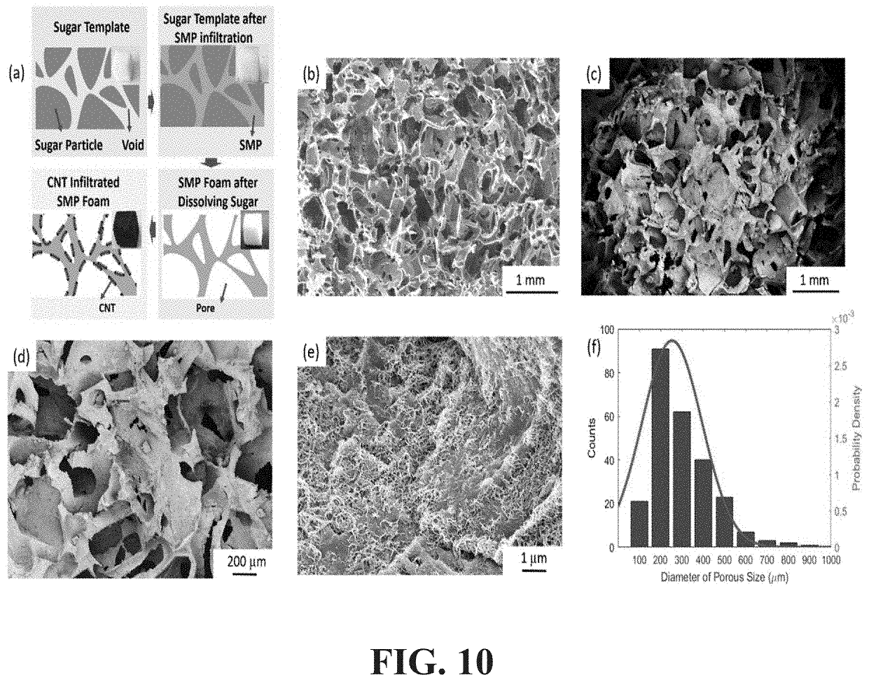

The shape recovery capability of pristine SMP foam is shown in FIG. 11, panel (a). A cubic SMP foam sample was compressed at 60° C. and cooled back to room temperature in the compressed shape. Then the cooled, compressed SMP sample was placed on a hot plate at 60° C. When the SMP temperature increased above its T_{g}, the sample autonomously changed shape and returned to its original, pre-compression shape. Due to its high porosity and flexibility, the SMP foam was able to recover from a more than 50% compressive strain.

As noted, the electrical conductivity of the CNT-SMP nanocomposite foams was enhanced by infiltrating CNTs into the porous SMP microstructure in the ethanol solution using ultrasonication. FIG. 11, panel (b) shows the electrical resistivity of the fabricated CNT/SMP nanocomposites. Both the CNT concentration and ultrasonication time had a significant impact on the electrical resistivity of the fabricated nanocomposites. It is noted that the prolonged ultrasonication time can reduce the electrical resistivity by more than 90%. For instance, the electrical resistivity of the nanocomposites was reduced from 2766 Ωm to 220 Ωm when the ultrasonication time was extended from 10 minutes to 50 minutes using a 0.001 g/ml CNT/ethanol solution. A similar electrical resistivity reduction was recorded from 573 Ωm to 47 Ωm using 0.006 g/ml CNT-ethanol solution. The CNT concentration used for nanocomposite fabrication was 0.005 g/ml for further studies. The ultrasonication time was 50 minutes.

Actuation of shape recovery is necessary for the SMP in ICA applications due to complex biological system requirements. A Joule-heating based SMP actuation mechanism was developed. To validate the Joule-heating based triggering mechanism, an electrically conductive carbon wire was inserted into a cubic CNT-SMP nanocomposite sample. The sample surface temperature was measured when the electrical current was set at 0.05-0.2 A. Insufficient heat was generated, and the sample surface temperature was kept at temperature below the T_{g} of SMP when 0.05 A and 0.1 A current was applied, respectively (FIG. 11, panel (c)). However, the sample surface temperature was able to quickly increase above the T_{g} when 0.15 A or higher current was applied during the Joule-heating process. It is noted that it took less than one minute to increase sample temperature above T_{g} and trigger the shape recovery of deformed nanocomposites. More accurate control of SMP shape recovery can be obtained by altering the electrical current during the deployment. Thus, the Joule-heating method used herein can be used in intracranial aneurysm applications using the presently disclosed porous nanocomposites.

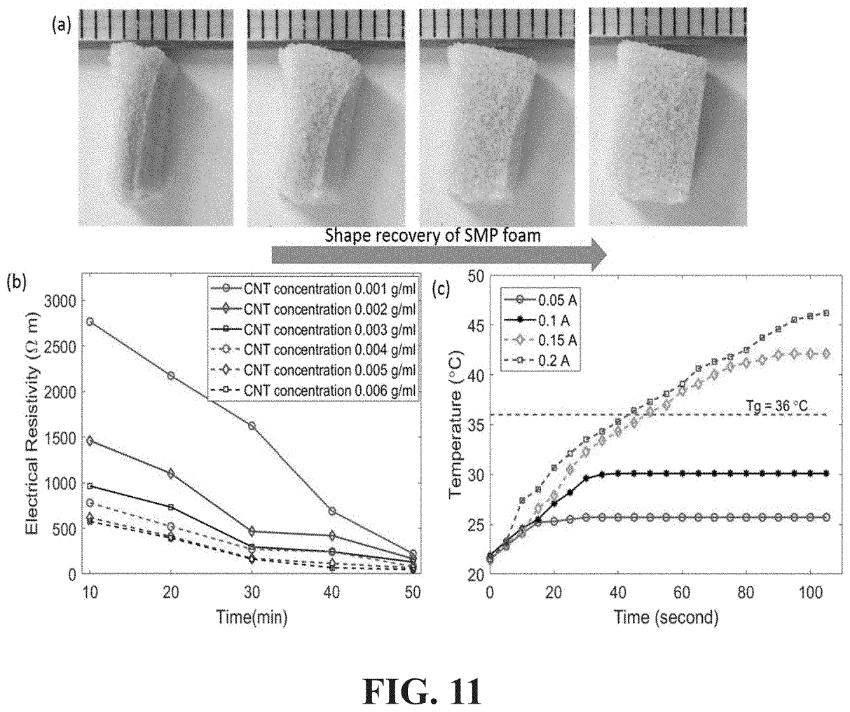

FIG. 12 shows the shape recovery of a CNT-SMP nanocomposite when 0.2 A electrical current was used in the Joule-heating method. A nanocomposite cubic sample was first compressed by more than 50% strain. Then, the 0.2 A electrical current was applied and the temperature of the compressed SMP nanocomposite sample increased above the T_{g} in 45 seconds, and the entire shape recovery took less than two minutes. The maximum surface temperature recorded from the SMP sample was 46.8° C., which was significantly lower than the polymer decomposition temperature of 260° C. Therefore, the heat generated by the Joule-heating method didn't cause any polymer decomposition and would be considered biologically safe for intracranial aneurysm applications.

These results demonstrate use of a highly porous CNT-SMP nanocomposite for the development of a novel SMP device to treat ICAs. As explained, the pristine SMP foam was first fabricated using a biologically safe sugar template, and then CNTs were infiltrated into the open cell foam using ultrasonication during nanocomposite fabrication. Uniform CNT distribution in the manufactured nanocomposites was validated by SEM microscopy. Both CNT concentration in ethanol solution and ultrasonication time are fabrication parameters that can be altered to vary the electrical properties of the compositions. The disclosed novel SMP nanocomposites exhibit good shape recovery capability triggered by the Joule-heating mechanism, recovering from the deformation of more than 50% compressive strain to their original shape within two minutes.

### Example III

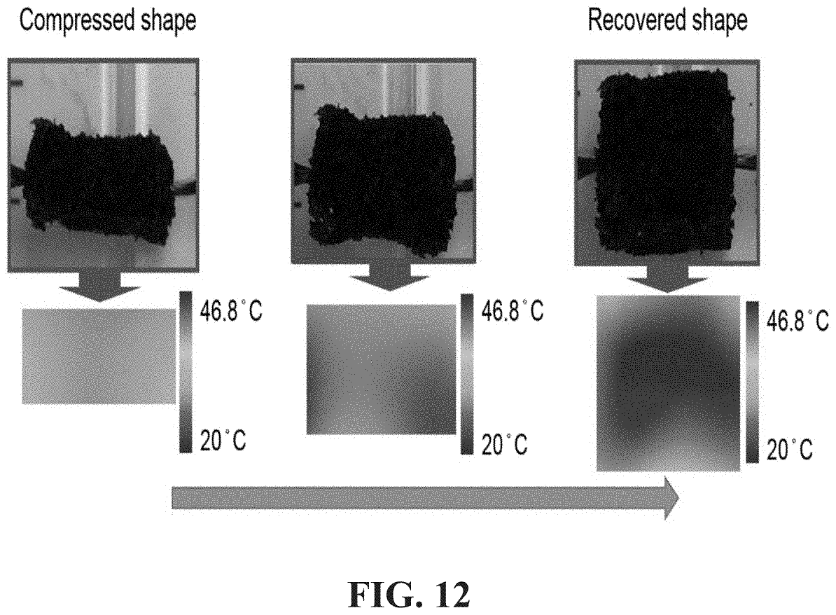

One non-limiting embodiment of the present disclosure is directed to a personalized SMP embolization device tailored to a patient's particular aneurysmal condition for treatment of an ICA. The SMP device is based on the specific geometry of the subject's aneurysm geometry. The device can be fabricated using additive manufacturing technology (e.g., 3D printing) to achieve a short preparation time before an operation/surgery. Image Data can be obtained, for example but not by way of limitation, via computed tomography (CT) or CT angiography (CTA). Semi-automatic image segmentation and 3D geometry reconstruction is used to investigate the patient-specific aneurysm environment (FIG. 13, panels (a-b)).

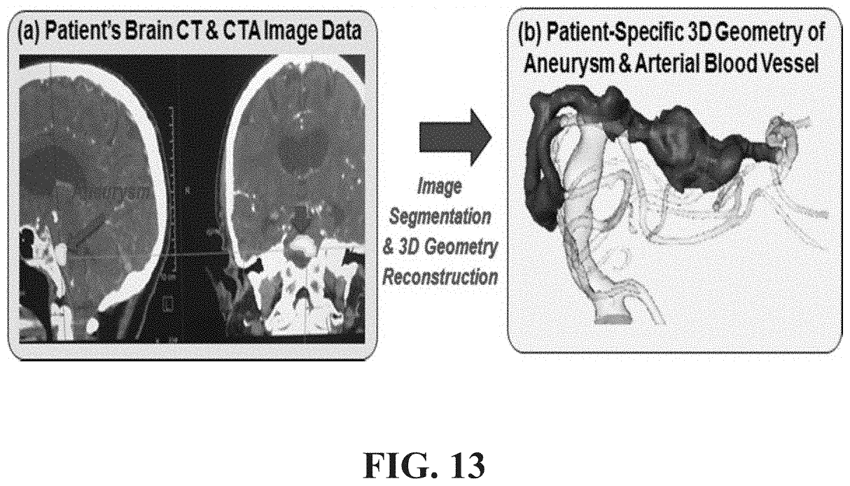

The SMP compositions that can be used herein include, but are not limited to, the aliphatic urethane SMP compositions described elsewhere herein, such as (but not limited to) polyurethanes made from high molecular monomers in various molar ratios, including HDI, HPED, and TEA (FIG. 14). By adjusting the weight ratios of the three monomers, the glass transition temperature (T_{g}), an important parameter for governing the shape changing of the synthesized SMPs, can be controlled in the range of 33° C. to 86° C. In at least certain non-limiting embodiments of the present disclosure, the glass transition temperature is targeted to be in a range of from about 36° C. to about 46° C., including about 37° C., about 38° C., about 39° C., about 40° C., about 41° C., about 42° C., about 43° C., about 44° C., about 45° C., and about 46° C. (and fractional values of such temperatures), which is slightly higher than the physiological human body temperature. The targeted mechanical properties, such as (but not limited to) the elastic modulus and failure stress/strain, can also be modified and tuned.

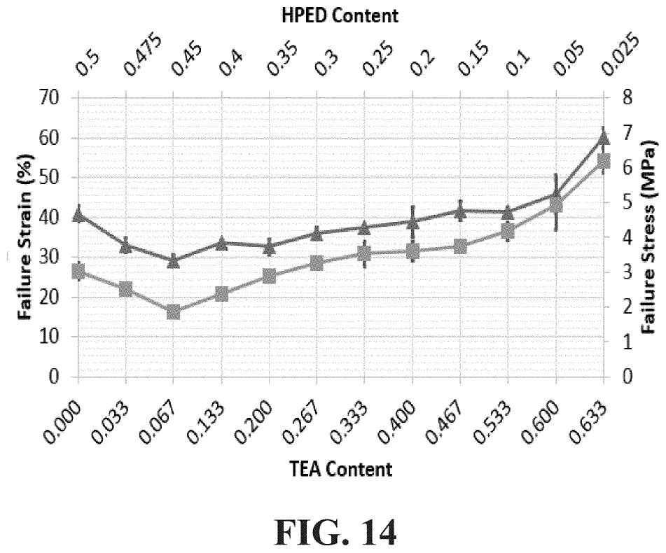

Cyclic uniaxial tensile tests were extensively carried out using ASTM D638 “dog-bone”-shaped specimens (length=9.5 mm, gauge width=3.2 mm, and thickness=2.4 mm) to determine the suitable mechanical strength and resistance. In brief, specimens were tested in a dual-column Instron system. Each specimen was clamped in the thermal chamber and kept at 10° C. above the T_{g} for 10-15 minutes before displacement-controlled cyclic tensile testing. Results demonstrated initial hysteresis corresponding to the residual stress; in addition, the results showed that once the SMPs have been fully stretched and relaxed for 3 cycles, the hysteresis characteristic becomes relatively insignificant (FIG. 15, panel (a)). Next, to simulate the mechanical behaviors of the synthesized SMPs under nonlinear finite element modeling framework, the Arruda-Boyce model, based on the statistical mechanics theory of polymeric substances, was employed. A strain energy density function W can be expressed by:

$$W ( {I}_{1} ) = {C}_{1} [ \frac{1}{2} ( {I}_{1} - 3 ) + \frac{1}{20 {λ}^{2}} ( {I}_{1}^{2} - 9 ) + \frac{\text{11}}{\text{1050} {λ}^{4}} ( {I}_{1}^{3} - 27 ) + \frac{19}{\text{7000} {λ}^{6}} ( {I}_{1}^{4} - 81 ) + \frac{519}{\text{673750} {λ}^{8}} ( {I}_{1}^{5} - 243 ) ] , \qquad ( Eqn . 3 )$$

where I₁ is the first invariant of the right Cauchy-Green deformation tensor, C₁ and λ are the material parameters denoting the strength of the polymer chains and their limiting network stretch, respectively. By implementing this constitutive model to a user material subroutine VUMAT in the FE software ABAQUS (Simulia, Dassault Systemes) and formulating an inverse modeling-based parameter estimation pipeline, two model parameters for the synthesized SMP materials have been quantified based on nonlinear fitting to the measured mechanical data using the Levenberg-Marquardt algorithm (FIG. 15, panel (b)).

Aneurysm Creation Model for In Vivo Small Animal Study

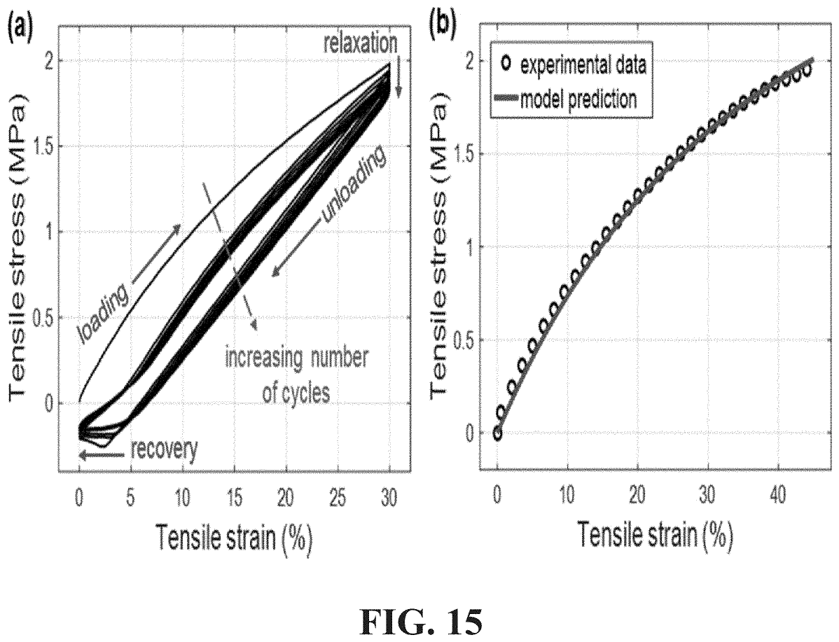

Aneurysm creation models in rabbits have been established as a useful means to evaluate the efficacy and performance of new endovascular embolization devices. An animal study was carried out using a modified elastase-induced aneurysm creation model (FIG. 16, panel (a)). Four weeks following the aneurysm creation (AC) procedure, an intravenous aortogram was used to evaluate aneurysm patency. The study showed the successful creation of an aneurysm in the right common carotid artery (FIG. 16, panel (b)).

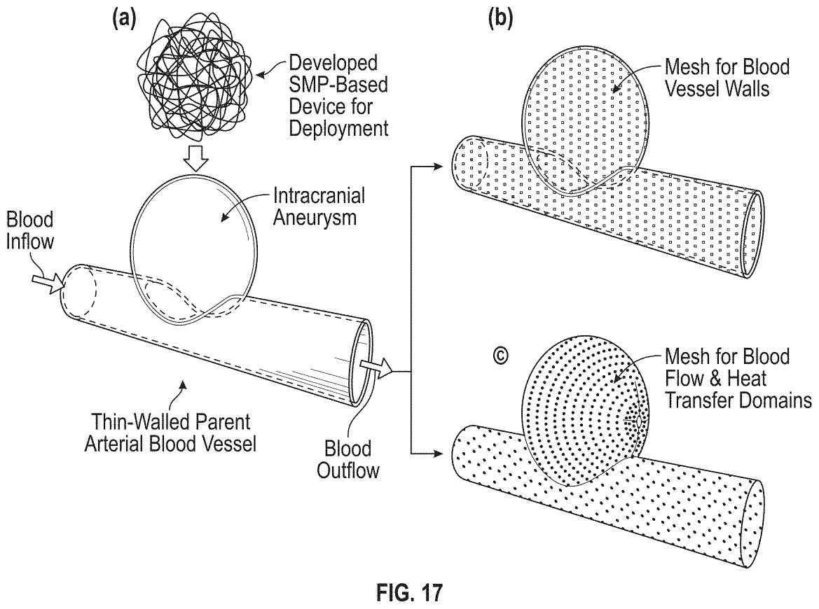

Using the acquired retrospective patient's brain image data, image segmentation is performed for reconstructing a high-fidelity aneurysm geometry and the surrounding arterial vessel environment. Patient-specific FE models perform endovascular simulations for assessing the hemodynamics and biomechanics of the deployed embolic devices. The FE computational models are validated against the measured flow data from an in-house in vitro flow loop with a particle image velocimetry (PIV) system (FIG. 18).

Image Segmentation & Geometry Reconstruction

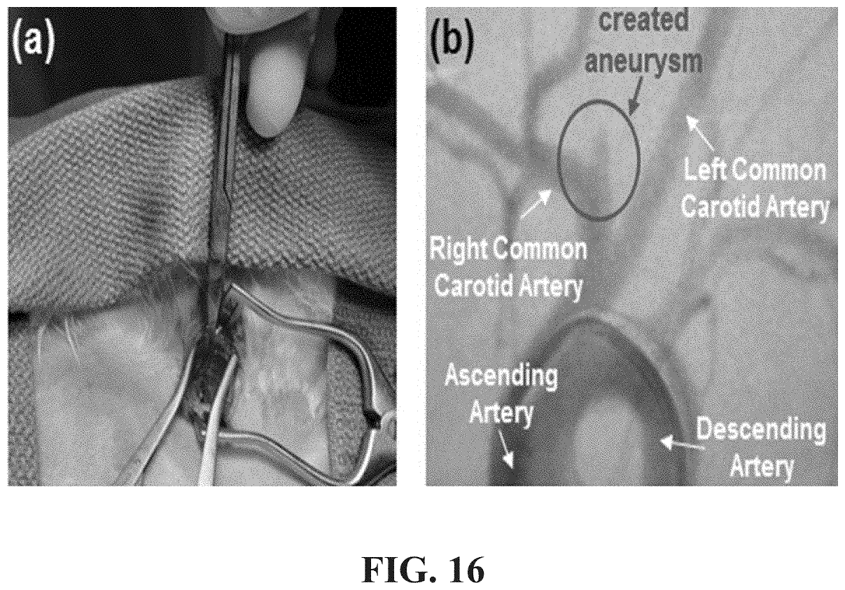

By using the patient's acquired CT angiography (CTA) DICOM image data, semi-automatic image segmentation, based on the developed pipeline, is performed with the image segmentation software Amira (FEI Inc., Hillsboro, Oreg.) to reconstruct high-fidelity 3D geometries of the patient arterial blood vessel and the aneurysm from each patient's brain image data (FIG. 13). Such patient-specific geometry (with an aneurysm height of 6.5-10.5 mm, an aneurysm width of 3.5-5.5 mm, and an aneurysm neck of 3.5-4.5 mm) is then employed for the development of patient-specific predictive FE models to simulate hemodynamic and thermal-mechanical behaviors of the arterial vessel and aneurysm environment in response to the deployed embolic devices (FIG. 17, panels (a-c)).

FSI and Thermal-Mechanical FE Simulation Framework

The reconstructed parent blood vessel and aneurysm's geometries are imported to the FE mesh generation software Hypermesh (Altair Engineering, Troy, Mich.) to obtain the meshes of both the structural domain (parent arterial vessel wall and aneurysm wall) and the fluid and heat transfer domain (FIG. 17, panels (b-c)). Then, constitutive models developed for the SMP materials are utilized to incorporate the thermal-mechanical responses for numerical investigations of the shape change triggering and device deployment processes.

Fabrication of Pristine SMP and SMP-Carbon Nanotubes (CNT) based Embolic Devices with Shape Change Triggering Element

Synthesized SMP foams and balloons are compressed into thin wires as the embolic device during fabrication. The pre-compressed shape autonomously recovers to fit into the 3D geometry of the ICA once it is heated up to the programmable temperature. The design and manufacture of these embolic SMP devices is described in more detail below.

SMP Materials, Shape Change Triggering Mechanisms, and Embolic Device Forms for Endovascular Embolization Applications

In non-limiting embodiments, three general types of SMP materials are used in the fabrication/manufacturing process: 1) pristine SMP integrated with a photo-thermal activation shape changing mechanism (with shape changing activation thermal energy converted from laser source via laser dye); 2) SMPs infused with CNTs (CNT-SMP nanocomposites) integrated with an electro-thermal activation mechanism (with activation thermal energy converted from electrical energy via electrically conductive carbon nanotubes); and 3) photo-thermally activated SMP devices equipped with a laser-based activation mechanism (with an appropriate wavelength of laser delivered by embedded optical fiber). In addition, three embolic device forms are fabricated: 1) coils, 2) open-cell foams, and 3) thin-shell balloons.

In-Vitro Experiments

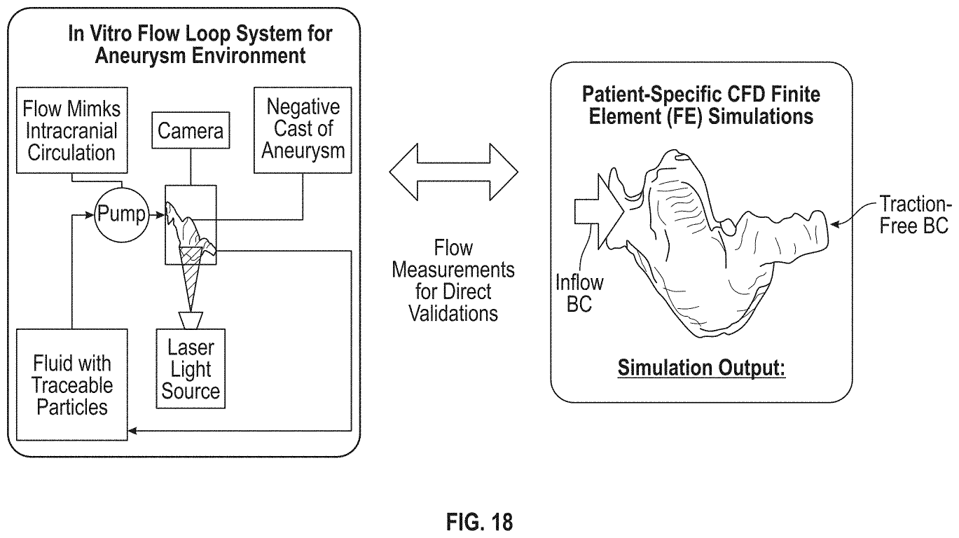

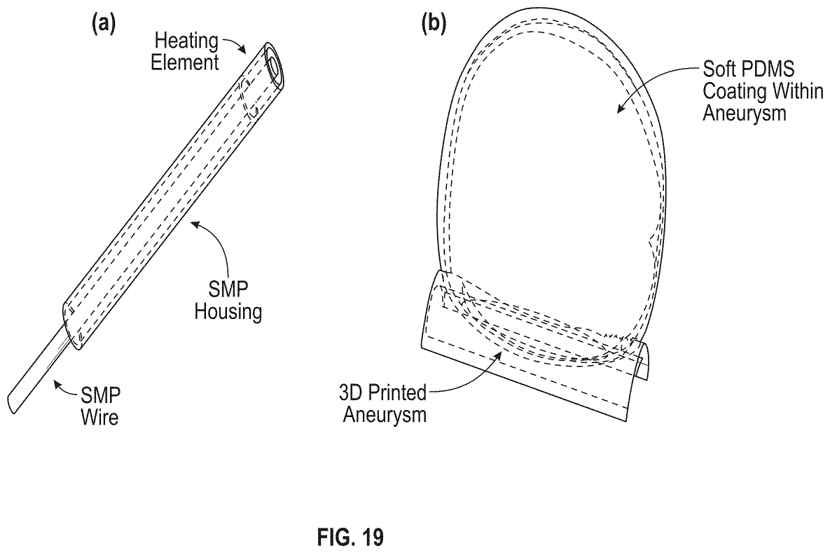

As noted above, SMP devices are manufactured based on a patient's brain image data by using a UV cured 3D-printing system and are then inserted into the aneurysm area (FIGS. 19-20). The fabricated model is cleaned following standard 3D printing procedures to obtain a smooth surface for implantation. Since the aneurysm is soft in nature, a coating layer made of soft polydimethylsiloxane (PDMS) is applied to the SMP device. The prepared SMP embolic device and the tube housing is then inserted into the aneurysm neck area. The heating element at the end of SMP housing is then activated. Once the temperature increases above the T_{g} of the SMP device, the device is slowly delivered out of the tube housing and starts to change shape, restoring to its programmed, pre-compression shape to fill in the aneurysmal space. The aneurysm's occlusion completeness can then be assessed by using the micro-CT scanner (Quantum FX System, PerkinElmer, Inc., Waltham, Mass.).

In certain non-limiting embodiments, the SMP devices are constructed according to the design parameters shown in Table 5.

TABLE 5
Design Criteria for SMP-Based Embolic Device Prototypes

| | Criterion | | Consideration | |
|---|---|---|---|---|
| T_{g} | | | 37° C.-43° | C. |
| | Elastic | (below T_{g}) | 0.5-2.0 | MPa |
| | Modulus | (above T_{g}) | 0.25-1.5 | MPa |
| | Tensile | (below T_{g}) | 2.5-3.5 | MPa |
| | Strength | (above T_{g}) | 2.0-2.75 | MPa |

| | |
|---|---|
| Tensile Failure Strain | >50% |
| Visible in CTA | ✓ (Min. Req.) |
| Visible in MRA | ✓ (More Ideal) |

### Example IV

Further analyses were performed to characterize the presently disclosed SMP materials. The aliphatic urethane SMP devices used in this Example were fabricated using the same monomeric materials disclosed in the above experiments, i.e., HDI, HPED, and TEA, combined in a molar ratio of 1:0.05:0.6 (HDI:HPED:TEA). To synthesize the solid SMPs, appropriate amounts of monomers were first measured and mixed using a high-speed shear mixer for three (3) minutes and then cast into “dog-bone” or rectangular molds for curing. The materials were degassed three times, and nitrogen was used to protect all the materials before and during curing using a vacuum oven. The temperature profile was first kept at room temperature for 60 minutes, followed by a ramp of 30° C. per hour up to 130° C., then followed by 1 hour at 130° C., and finally cooled back to room temperature naturally. The fully cured SMP samples were removed from the molds and saved for testing and characterization.

SMP foams were synthesized using a sugar-assisted method. A sugar bar was first manufactured by compressing an appropriate amount of cane sugar (sucrose) into a silicon rubber mold. A slight amount of water was added to improve the formability of cane sugar. Once the sugar bar was prepared, appropriate amounts of the monomers were measured and combined and mixed by a high-speed shear mixer for 3 minutes. The sugar bar was then merged into the monomer mixture, and was kept in an ice bath in vacuum for 24 hours. The monomers were partially reacted, and the bar was post-cured following the heating procedure introduced above. The fully cured SMP/sugar bar was then merged in de-ionized water and kept in a bath sonicator for 1 hour to fully dissolve cane sugar. The manufactured SMP foams were kept in a vacuum oven at 30° C. for 24 hours to fully eliminate all the humidity trapped in the SMP foams.

Experimental Characterization of Synthesized SMP

Dynamic mechanical analysis (DMA) was employed to determine the thermal transitions in the synthesized SMPs. Experiments were conducted using a TAQ800 dynamic mechanical analyzer. Rectangular beam samples were tested using tensile mode from 30° C. to 130° C. Differential scanning calorimetry (DSC) was employed to characterize the polymer's thermal properties, in particular to validate the glass transition temperature (T_{g}). The comparison of T_{g}'s obtained from DMA and DSC can validate each other. Thermogravimetric analysis (TGA) tests were conducted to study the decomposition temperature and procedure of the synthesized SMP. The maximum allowable temperature of the SMP can be obtained from the TGA tests.

Microscale Imaging Using SEM

Scanning electron micrography (SEM) imaging was employed to visualize the porous size distribution within the SMP foam. The SEM images were taken from two different layers of an SMP foam to investigate the average size of SMP at different locations.

Results

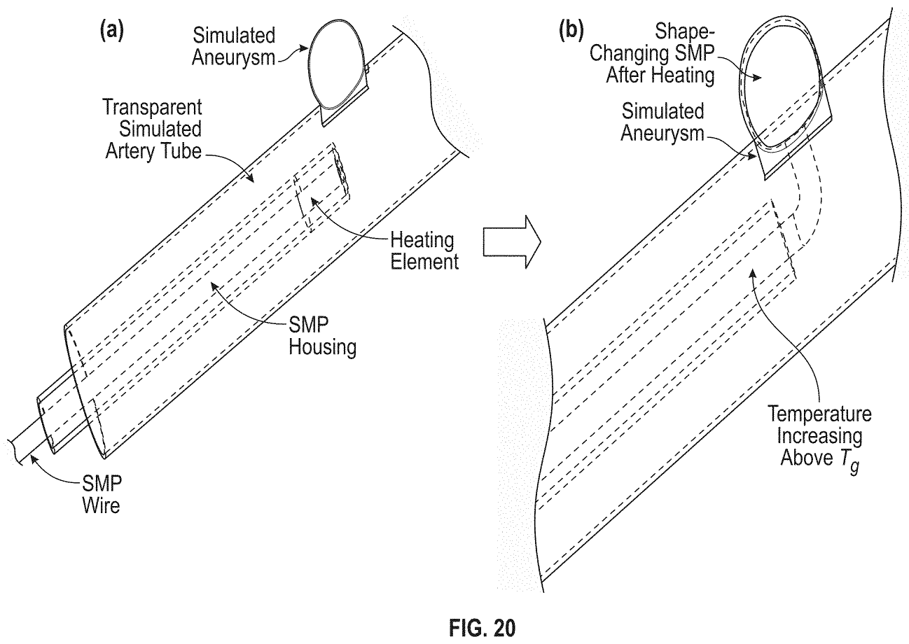

The shape memory functions of the synthesized SMPs were investigated. Each straight beam sample was first immersed into hot water (at 5-10° C. above the expected glass transition temperature), bent up to 180° to form a bended shape, then cooled back to room temperature while maintaining the bended shape. Then the SMP sample was placed back into hot water and the material restored to the initial straight form autonomously. SMP samples at different shapes are illustrated in FIG. 21.

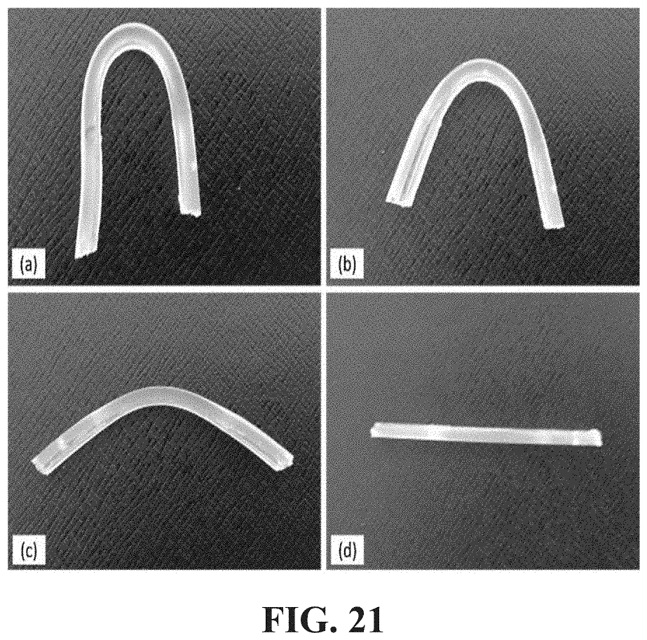

DMA tests were applied to evaluate the T_{g} of the SMP. As shown in FIG. 22, the glass transition temperature of synthesized polymer is the temperature where tan (δ) reached the peak value. The measured glass transition temperature of the SMPs was 39° C., a temperature slightly above normal body temperature.

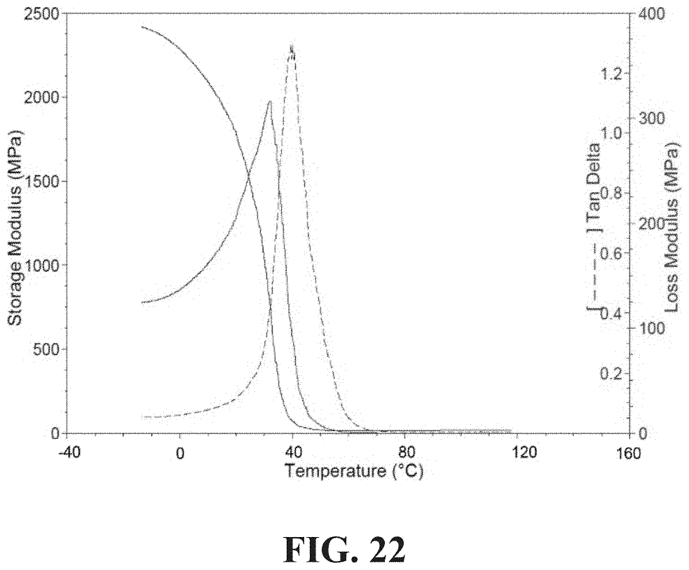

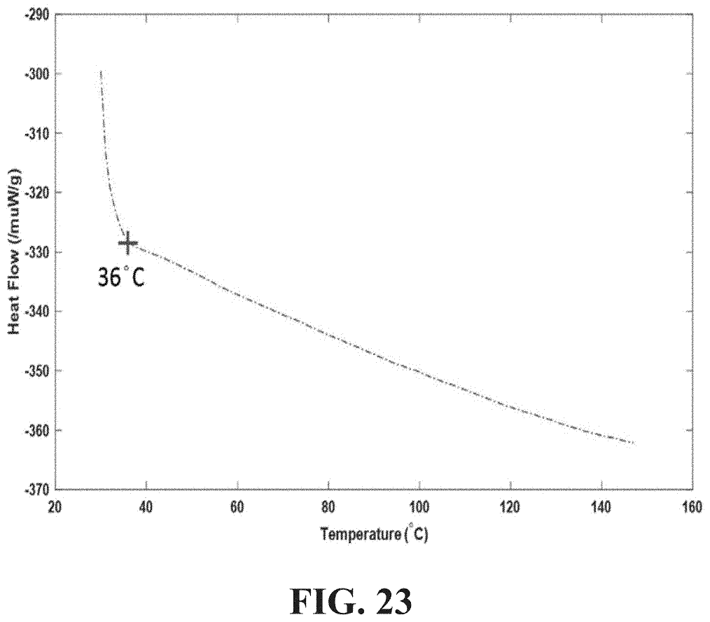

DSC testing results are shown in FIG. 23. The obtained T_{g} was 36° C. It is normal to have a slight difference between the results of DSC and DMA tests due to the different testing mechanisms. The DSC experimental results validated that the T_{g} of synthesized SMP is close to normal body temperature. TGA testing results is shown in FIG. 24. It is noted that the synthesized SMP started decomposing at 247° C. This means that the maximum polymer temperature allowed on the developed SMP should be significantly less than the measured temperature. This information is useful for the control of electrical DC current during the Joule-heating process. More details will be discussed below.

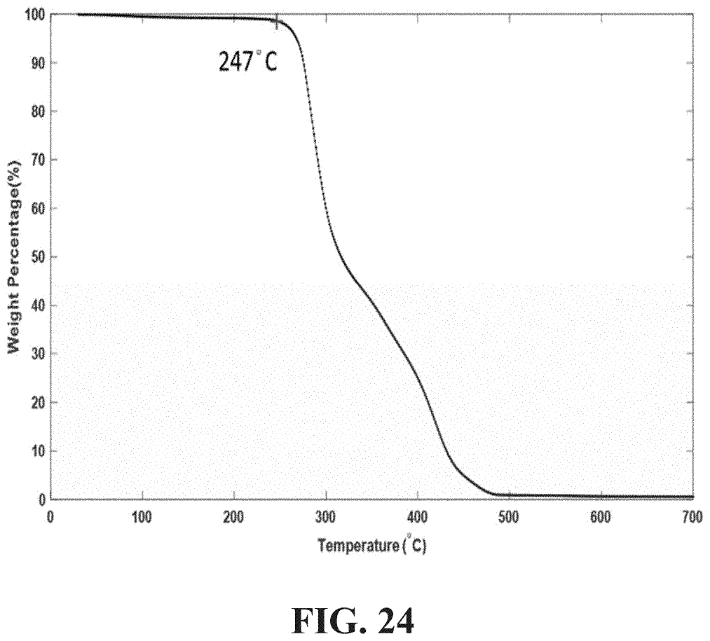

Once the SMP solid samples were fully characterized, more detailed characterizations were carried out to understand the properties of the SMP foams. The fabricated SMP foams were examined using a SEM to measure the size of cells for evaluating the density and compression capability of the synthesized SMP foams. The SEM images were taken from two different layers of a single SMP foam sample. The typical SEM images are shown in FIG. 25. The average cell size is around 500 μm.

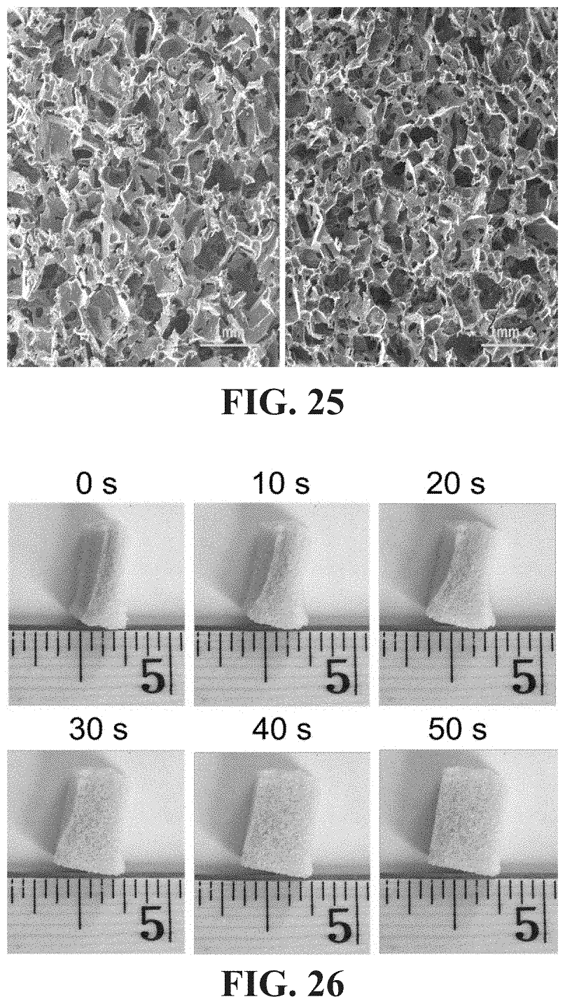

The shape recovery capability of the SMP foams was first obtained using a direct heating method. The SMP foam sample was first compressed and kept in a refrigerator at 5° C. to keep the deformed shape. Then the sample was placed on a hot plate with a surface temperature of 70° C. As shown in FIG. 26, the SMP foam started to recover from the deformed and compressed shape after 10 seconds. In 50 seconds, the shape of the SMP foam had completely recovered back to a normal cubic geometry. The fast shape recovery demonstrated that the presently disclosed SMP foams can recover from deformation within a time span suitable for biomedical applications such as (but not limited to) ICA treatment.

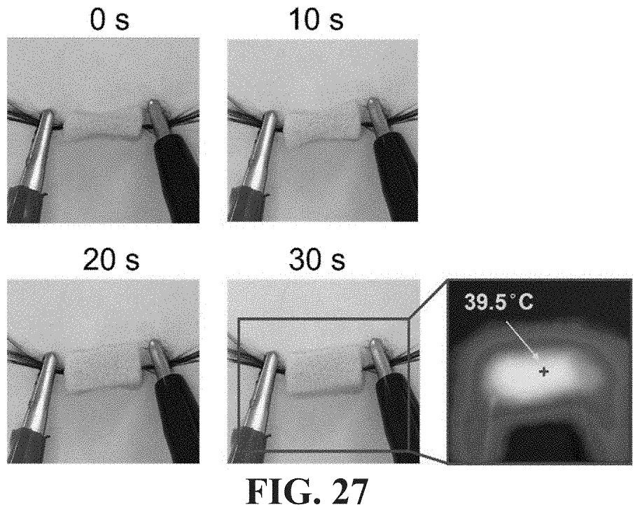

Direct heating is not easy to implement, in particular for FDA approval. Therefore, a Joule-heating based approach was employed. As shown in FIG. 27, a DC current of 0.2 A was able to generate sufficient heat so that the SMP foams were able to fully recover from the compressed shape to the original shape in 30 seconds.

### Example V

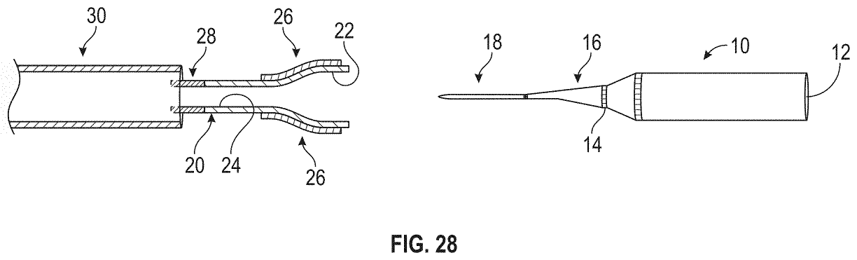

In certain non-limiting embodiments, the present disclosure is directed to an SMP device, such as (but not limited to) an SMP sponge, compressed into a wire or filament for delivery in a medical/surgical application by a catheter, and a method of such compression. As shown in FIG. 28, an SMP porous wire 10 is used as an example to demonstrate the compression process. The SMP porous wire 10 has a first end 12 and a second end 14, and the SMP porous wire is first compressed at the second end 14 by a metal clamp 16, and the second end 14 of the SMP porous wire 10 is compressed. A needle 18 connected to the metal clamp 16 can lead the SMP porous wire 10 into a thin metal tube 20. Then the needle 18, metal clamp 16, and SMP porous wire 10 are slowly inserted into the metal tube 20. A first end 22 of the metal tube 20 is larger than a second end 24 thereof to allow the entrance of the SMP porous wire 10 into the metal tube 20 without any cutting or damage to the SMP porous wire 10. In addition, the first end 22 of the metal tube 20 can be heated to 45-50° C. (e.g., 5° C.-10° C. above the glass transition temperature of the SMP porous wire) by a heating element 26 (comprising, for example but not by way of limitation, an electrothermal, photothermal, or heat resistive mechanism) surrounding the metal tube 20. The SMP porous wire 10 is heated and softened when it passes this area. The softened SMP porous wire 10 is further compressed as it passes through the thin metal tube 20 to the second end 24 thereof, where it is cooled back to room temperature in a cooling section 28 of the tube 20 to lock the SMP porous wire 10 into the compressed shape. Finally, the compressed SMP porous wire 10 is inserted into a catheter 30, and the metal needle 18 and metal clamp 16 are removed from the catheter 30, leaving the compressed SMP porous wire 10 in the catheter 30 for a later medical/surgical application. Due to the large amount of deformation, the SMP sponge can carry in a compressed state, other shapes of the SMP sponge can be compressed and inserted into a catheter using a similar method. The shape of the metal tube used for compression can be adjusted accordingly.

It will be understood from the foregoing description that various modifications and changes may be made in the various embodiments of the present disclosure without departing from their true spirit. The description provided herein is intended for purposes of illustration only and is not intended to be construed in a limiting sense, except where specifically indicated. Thus, while the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the apparatus, formulations of the various components and compositions described herein, the methods described herein, or in the steps or the sequence of steps of the methods described herein without departing from the spirit and scope of the present disclosure.

What is claimed is:

1. A shape memory polymer (SMP) device, comprising:
an SMP material having a permanent shape, a temporary shape, and a glass transition temperature, wherein the SMP material when in the permanent shape has a specific three-dimensional (3D) geometry unique to a specific intracorporeal defect in a subject, such that the permanent shape of the SMP material will substantially conform to and fill a space in the specific intracorporeal defect in the subject when the SMP material is deployed into the specific intracorporeal defect at a temperature above the glass transition temperature of the SMP material.

2. The SMP device of claim 1, wherein the intracorporeal defect is an aneurysm.

3. The SMP device of claim 2, wherein the aneurysm is an intracranial aneurysm (ICA).

4. The SMP device of claim 1, wherein the glass transition temperature is in a range from about 36° C. to about 46° C.

5. The SMP device of claim 1, wherein the glass transition temperature is in a range from about 37° C. to about 43° C.

6. The SMP device of claim 1, wherein the 3D geometry of the permanent shape of the SMP material is obtained from computed tomography (CT) imaging of the specific intracorporeal defect in the subject.

7. The SMP device of claim 1, wherein the SMP material is a 3D-printed SMP object.

8. The SMP device of claim 1, wherein the SMP material is an open-cell material comprising pores, and wherein the pores are coated with a blood coagulant.

9. The SMP device of claim 1, wherein the SMP material has an external surface, and wherein at least a portion of the external surface is coated with a blood anticoagulant.

10. The SMP device of claim 1, wherein the SMP material comprises Hexamethylene diisocyanate (HDI), N,N,N0,N0-tetrakis (hydroxypropyl) ethylenediamine (HPED), and Triethanolamine (TEA).

11. The SMP device of claim 1, wherein the SMP material comprises a radio-opaque additive.

12. The SMP device of claim 1, wherein the SMP material is a carbon nanotube (CNT)-SMP material.

13. An SMP device delivery system, comprising:
the SMP device of claim 1, wherein the SMP device is in its temporary shape; and
a heating mechanism for raising the temperature of the SMP device to a temperature above the glass transition temperature of the SMP material before the SMP device is deployed into the specific intracorporeal defect of the subject.

14. The SMP device delivery system of claim 13, wherein the heating mechanism is electrothermal.

15. The SMP device delivery system of claim 13, wherein the heating mechanism is photothermal.

16. The SMP device delivery system of claim 13, wherein the heating mechanism is heat resistive.

17. The SMP device delivery system of claim 13, further comprising a catheter for delivery of the SMP device into the specific intracorporeal defect in the subject.

18. The SMP device delivery system of claim 17, wherein the heating mechanism comprises a portion of a terminal end of the catheter.

19. A method of treating an intracorporeal defect in a subject, comprising:
inserting the SMP device of claim 1 into the intracorporeal defect of the subject.

20. The method of claim 19, wherein the intracorporeal defect is an aneurysm.

21. The method of claim 20, wherein the aneurysm is an intracranial aneurysm (ICA).

22. A method of delivering an SMP device into an intracorporeal defect in a subject, the method comprising:
inserting the SMP delivery system of claim 13 into the subject to deliver the SMP device into the intracorporeal defect.

23. The method of claim 22, wherein the intracorporeal defect is an aneurysm.

24. The method of claim 23, wherein the aneurysm is an intracranial aneurysm (ICA).
